(19) United States
(12) Patent Application Publication
(10) Pub. No.: US 2025/0258162 A1
(43) Pub. Date: Aug. 14, 2025

(54) REGULATORY T CELL STABILITY ASSAY

(71) Applicant: Abata Therapeutics, Inc., Watertown, MA (US)

(72) Inventors: Yanbo ZHANG, Watertown, MA (US); Devapregasan MOODLEY, Watertown, MA (US)

(21) Appl. No.: 19/181,950

(22) Filed: Apr. 17, 2025

Related U.S. Application Data
(63) Continuation of application No. PCT/US2023/077260, filed on Oct. 19, 2023.
(60) Provisional application No. 63/432,136, filed on Dec. 13, 2022.
(60) Provisional application No. 63/417,474, filed on Oct. 19, 2022.

(51) Int. Cl. G01N 33/50; C12N 5/00; C12N 5/0783

(52) U.S. Cl. CPC G01N 33/505; C12N 5/0018; C12N 5/0637; G01N 33/5023; C12N 2500/33; C12N 2501/15; C12N 2501/2301; C12N 2501/2302; C12N 2501/2306; C12N 2501/2312; C12N 2501/2317; C12N 2501/24; C12N 2501/25; C12N 2501/51; C12N 2501/515; C12N 2501/998; G01N 2333/4703; G01N 2333/535; G01N 2333/70514; G01N 2333/7155

(57) ABSTRACT

The present disclosure is directed, in some aspects to methods and compositions for the assessment of regulatory T cell stability over time. In other aspects, the present disclosure provides methods and compositions for expanding regulatory T cells in culture.

### RELATED APPLICATIONS

The present application is a continuation of International Application No. PCT/US2023/077260, filed Oct. 19, 2023, which claims priority to U.S. provisional application No. 63/417,474, filed Oct. 19, 2022, and U.S. provisional application No. 63/432,136, filed Dec. 13, 2022, the contents of each of which are incorporated by reference herein in its entirety.

### BACKGROUND

Regulatory T cells have potential for the treatment of diseases, such as autoimmune diseases, because they can target specific diseased cell types. Regulatory T cells can be useful, for example, in generating a local immune response that is specific to certain cell types and tissues associated with a disease of interest via an antigen-specific mechanism. Nonetheless, robust clinical scale and clinical grade manufacturing of stable antigen-specific regulatory T cells has been challenging. Thus, new methods of manufacturing, characterizing, culturing and expanding such regulatory T cells are needed.

### SUMMARY

Some aspects of the disclosure provide a method of evaluating the stability profile of an isolated cell population comprising regulatory T cells. In some embodiments, a method of evaluating the stability profile of an isolated cell population comprising regulatory T cells comprises: (i) culturing the isolated cell population in the presence of two or more pro-inflammatory cytokines for a period of time; (ii) characterizing one or more markers of stability of the isolated cell population following the period of time; and (iii) evaluating the stability profile of the isolated cell population based on the characterizing of the one or more markers of stability.

Some aspects of the disclosure provide a method comprising (i) culturing an isolated cell population comprising regulatory T cells in the presence of two or more pro-inflammatory cytokines for a period of time; and (ii) characterizing one or more markers of stability of the isolated cell population following the period of time.

In some embodiments, the two or more pro-inflammatory cytokines comprises at least two, at least three, at least four, at least five, at least six, at least seven, at least eight, or at least nine cytokines selected from the group consisting of Interleukin-2 (IL-2), Interleukin-6 (IL-6), Interleukin-12 (IL-12) (also known as IL-12p70), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

In some embodiments, the two or more pro-inflammatory cytokines comprises IL-2 and at least one, at least two, at least three, at least four, at least five, at least six, at least seven, or at least eight cytokines selected from the group consisting of IL-6, IL-12, TNF-α, IL-17A, IFN-β, IFN-γ, TGF-β1, and IL-1β.

In some embodiments, the two or more pro-inflammatory cytokines comprises: (a) IL-2 and IL-6; (b) IL-2 and IL-12; (c) IL-2 and TNF-α; (d) IL-2 and IL-17A; (e) IL-2 and IFN-β; (f) IL-2 and IFN-γ; (g) IL-2 and TGF-β1; or (h) IL-2 and IL-1β.

In some embodiments, the two or more pro-inflammatory cytokines comprises: (a) IL-2, IL-6, and at least one additional pro-inflammatory cytokine selected from: IL-12, TNF-α, IL-17A, IFN-β, IFN-γ, TGF-β1, and IL-1β; (b) IL-2, IL-12, and at least one additional pro-inflammatory cytokine selected from: IL-6, TNF-α, IL-17A, IFN-β, IFN-γ, TGF-β1, and IL-1β; (c) IL-2, TNF-α, and at least one additional pro-inflammatory cytokine selected from: IL-6, IL-12, IL-17A, IFN-β, IFN-γ, TGF-β1, and IL-1β; (d) IL-2, IL-17A, and at least one additional pro-inflammatory cytokine selected from: IL-6, IL-12, TNF-α, IFN-β, IFN-γ, TGF-β1, and IL-1β; (e) IL-2, IFN-β, and at least one additional pro-inflammatory cytokine selected from: IL-6, IL-12, TNF-α, IL-17A, IFN-γ, TGF-β1, and IL-1β; (f) IL-2, IFN-γ, and at least one additional pro-inflammatory cytokine selected from: IL-6, IL-12, TNF-α, IL-17A, IFN-β, TGF-β1, and IL-1β; (g) IL-2, TGF-β1, and at least one additional pro-inflammatory cytokine selected from: IL-6, IL-12, TNF-α, IL-17A, IFN-β, IFN-γ, and IL-1β; or (h) IL-2, IL-1β, and at least one additional pro-inflammatory cytokine selected from: IL-6, IL-12, TNF-α, IL-17A, IFN-β, IFN-γ, and TGF-β1.

In some embodiments, the two or more pro-inflammatory cytokines comprises: (a) IL-2, IL-6, and at least two additional pro-inflammatory cytokines selected from: IL-12, TNF-α, IL-17A, IFN-β, IFN-γ, TGF-β1, and IL-1β; (b) IL-2, IL-12, and at least two additional pro-inflammatory cytokines selected from: IL-6, TNF-α, IL-17A, IFN-β, IFN-γ, TGF-β1, and IL-1β; (c) IL-2, TNF-α, and at least two additional pro-inflammatory cytokines selected from: IL-6, IL-12, IL-17A, IFN-β, IFN-γ, TGF-β1, and IL-1β; (d) IL-2, IL-17A, and at least two additional pro-inflammatory cytokines selected from: IL-6, IL-12, TNF-α, IFN-β, IFN-γ, TGF-β1, and IL-1β; (e) IL-2, IFN-β, and at least two additional pro-inflammatory cytokines selected from: IL-6, IL-12, TNF-α, IL-17A, IFN-γ, TGF-β1, and IL-1β; (f) IL-2, IFN-γ, and at least two additional pro-inflammatory cytokines selected from: IL-6, IL-12, TNF-α, IL-17A, IFN-β, TGF-β1, and IL-1β; (g) IL-2, TGF-β1, and at least two additional pro-inflammatory cytokines selected from: IL-6, IL-12, TNF-α, IL-17A, IFN-β, IFN-γ, and IL-1β; or (h) IL-2, IL-1β, and at least two additional pro-inflammatory cytokines selected from: IL-6, IL-12, TNF-α, IL-17A, IFN-β, IFN-γ, and TGF-1.

In some embodiments, the two or more pro-inflammatory cytokines comprises: (a) IL-2, IL-6, and at least three additional pro-inflammatory cytokines selected from: IL-12, TNF-α, IL-17A, IFN-β, IFN-γ, TGF-β1, and IL-1β; (b) IL-2, IL-12, and at least three additional pro-inflammatory cytokines selected from: IL-6, TNF-α, IL-17A, IFN-β, IFN-γ, TGF-β1, and IL-1β; (c) IL-2, TNF-α, and at least three additional pro-inflammatory cytokines selected from: IL-6, IL-12, IL-17A, IFN-β, IFN-γ, TGF-β1, and IL-1β; (d) IL-2, IL-17A, and at least three additional pro-inflammatory cytokines selected from: IL-6, IL-12, TNF-α, IFN-β, IFN-γ, TGF-β1, and IL-1β; (e) IL-2, IFN-β, and at least three additional pro-inflammatory cytokines selected from: IL-6, IL-12, TNF-α, IL-17A, IFN-γ, TGF-β1, and IL-1β; (f) IL-2, IFN-γ, and at least three additional pro-inflammatory cytokines selected from: IL-6, IL-12, TNF-α, IL-17A, IFN-β, TGF-β1, and IL-1β; (g) IL-2, TGF-β1, and at least three additional pro-inflammatory cytokines selected from: IL-6, IL-12, TNF-α, IL-17A, IFN-β, IFN-γ, and IL-1β; or (h) IL-2, IL-1β, and at least three additional pro-inflammatory cytokines selected from: IL-6, IL-12, TNF-α, IL-17A, IFN-β, IFN-γ, and TGF-β1.

In some embodiments, the two or more pro-inflammatory cytokines comprises IL-2, IL-6, IL-12, TNF-α, IL-17A, IFN-β, IFN-γ, TGF-β1, and IL-1β.

In some embodiments, the two or more pro-inflammatory cytokines comprise IL-2, IL-1β, IL-6, IL-12, IL-17A, IFN-γ, TNF-α, IFN-β, and TGF-β1. In some embodiments, the concentration of IL-2 is about 1000 U/mL and/or the concentration of each of IL-1β, IL-6, IL-12, IL-17A, IFN-γ, TNF-α, IFN-β, and TGF-β1 is about 1000 pg/mL. In some embodiments, the concentration of IL-2 is about 1000 U/mL and the concentration of each of IL-1β, IL-6, IL-12, IL-17A, IFN-γ, TNF-α, IFN-β, and TGF-β1 is about 1000 pg/mL. the concentration of each of IL-1β, IL-6, IL-12, IL-17A, IFN-γ, TNF-α, IFN-β, and TGF-β1 is about 1000 pg/mL.

In some embodiments, the two or more pro-inflammatory cytokines comprise IL-2, IL-1β, IL-6, IL-12, IL-17A, IFN-γ, and TNF-α. In some embodiments, the concentration of IL-2 is about 1000 U/mL and/or the concentration of each of IL-1β, IL-6, IL-12, IL-17A, IFN-γ, and TNF-α is about 1000 pg/mL. In some embodiments, the concentration of IL-2 is about 1000 U/mL and the concentration of each of IL-1β, IL-6, IL-12, IL-17A, IFN-γ, and TNF-α is about 1000 pg/mL.

In some embodiments, the two or more pro-inflammatory cytokines further comprises Interleukin-22 (IL-22), Granulocyte-macrophage colony-stimulating factor (GM-CSF) and/or Interleukin-23 (IL-23). In some embodiments, the two or more pro-inflammatory cytokines further comprises Interleukin-22 (IL-22), Granulocyte-macrophage colony-stimulating factor (GM-CSF) and Interleukin-23 (IL-23).

In some embodiments, the concentration of any one of the pro-inflammatory cytokines (e.g., IL-2, IL-6, IL-12, TNF-α, IL-17A, IFN-β, IFN-γ, TGF-β1, and IL-1β) is in the range of 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

In some embodiments, the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL.

In some embodiments, the isolated cell population comprising regulatory T cells in the presence of two or more pro-inflammatory cytokines, as described herein, an anti-CD3antibody and an anti-CD28 antibody. In some embodiments, the anti-CD3 antibody and anti-CD28 antibody are attached to a solid support, e.g., a magnetic or polymeric bead. In some embodiments, the anti-CD3 antibody and anti-CD28 antibody are suspended in a matrix.

In some embodiments, the period of time is at least 3, at least 6, at least 9, at least 12, at least 15, at least 20, or at least 25 days. In some embodiments, the period of time is 3 days, 4 days, 5 days, 6 days, 7 days, 8 days, or 9 days. The period of time may be 2-30 days, 3-25 days, 5-25 days, 5-20 days, 5-10 days, 10-30 days, 10-25 days, 10-20 days, 15-30 days, or 20-30 days. In some embodiments, the period of time is about 3 days. In some embodiments, the period of time is about 4 days. In some embodiments, the period of time is about 5 days.

In some embodiments, the method further comprises characterizing the one or more markers of stability of the isolated cell population prior to (i).

In some embodiments, the method further comprises activating the isolated cell population between steps (i) and (ii). In some embodiments, activating the isolated cell population is performed by incubating the cell population with phorbol 12-myristate 13-acetate (PMA) and ionomycin. In some embodiments, activating the isolated cell population is performed by incubating the cell population with a mixture of anti-CD3 and/or anti-CD28 (e.g., anti-CD3 and anti-CD28) antibodies, e.g., polystyrene beads coated with a mixture of anti-CD3 and/or anti-CD28 (e.g., anti-CD3 and anti-CD28) antibodies.

In some embodiments, the method further comprises characterizing the one or more markers of stability of the isolated cell population at regular intervals throughout the period of time.

In some embodiments, the method comprises characterizing the one or more markers of stability of the isolated cell population every day, every second day, every third day, or every fourth day throughout the period of time.

In some embodiments, characterizing one or more markers of stability comprises determining expression and/or activity of the one or more markers of stability. In some embodiments, characterizing one or more markers of stability comprises determining expression of the one or more markers of stability. In some embodiments, characterizing one or more markers of stability comprises determining activity of the one or more markers of stability. In some embodiments, determining expression and/or activity of the one or more markers of stability is performed using biochemical techniques. In some embodiments, determining expression and/or activity of the one or more markers of stability is performed using fluorescence activated cell sorting (FACS).

The one or more markers of stability may be the biomarkers CD4, CD25, and/or FOXP3. In some embodiments, the biomarker of stability is CD4. In some embodiments, the biomarker of stability is CD25. In some embodiments, the biomarker of stability is FOXP3. In some embodiments, the one or more markers of stability comprise the methylation of a regulatory T cell-specific demethylation region (TSDR) at an endogenous FOXP3 locus and/or FOXP3 expression.

Some aspects of the disclosure provide a method of expanding a cell population comprising regulatory T cells.

In some embodiments, a method of expanding a cell population comprising regulatory T cells comprises culturing an isolated cell population comprising regulatory T cells in the presence of IL-2 and at least three additional pro-inflammatory cytokines.

In some embodiments, the at least three additional pro-inflammatory cytokines are selected from the group consisting of Interleukin-6 (IL-6), Interleukin-12 (IL-12), Tumor necrosis factor (TNF-α), Interleukin-17 (IL-17A), Interleukin-21 (IFN-β), Interferon gamma (IFN-γ), Transforming growth factor beta 1 (TGF-β1), and Interleukin-1b (IL-1β). In some embodiments, the at least three additional pro-inflammatory cytokine comprises at least four, at least five, at least six, at least seven, or at least eight additional pro-inflammatory cytokines selected from the group consisting of Interleukin-6 (IL-6), Interleukin-12 (IL-12), Tumor necrosis factor (TNF-α), Interleukin-17 (IL-17A), Interferon beta (IFN-β), Interferon gamma (IFN-γ), Transforming growth factor beta 1 (TGF-β1), and Interleukin-1b (IL-1β). In some embodiments, the at least three additional pro-inflammatory cytokines comprise IL-6, IL-12, TNF-α, IL-17A, IFN-β, IFN-γ, TGF-β1, and IL-1β.

Some aspects of the disclosure provide a method of expanding a cell population comprising regulatory T cells comprising culturing an isolated cell population comprising regulatory T cells in the presence of Interleukin-17 (IL-17A).

Some aspects of the disclosure provide a method of expanding a cell population comprising regulatory T cells comprising culturing an isolated cell population comprising regulatory T cells in the presence of Interferon beta (IFN-β).

Some aspects of the disclosure provide a method of expanding a cell population comprising regulatory T cells comprising culturing an isolated cell population comprising regulatory T cells in the presence of Interferon gamma (IFN-γ).

Some aspects of the disclosure provide a method of expanding a cell population comprising regulatory T cells comprising culturing an isolated cell population comprising regulatory T cells in the presence of Interleukin-1b (IL-1β).

Some aspects of the disclosure provide a method of expanding a cell population, the method comprising culturing an isolated cell population comprising regulatory T cells in the presence of Tumor necrosis factor alpha (TNF-α) and IL-2.

In some embodiments, a method of expanding a cell population comprising regulatory T cells further comprises culturing the isolated cell population in the presence of one or more additional pro-inflammatory cytokines. In some embodiments, one or more additional pro-inflammatory cytokines is Interleukin-2 (IL-2), Interleukin-6 (IL-6), Interleukin-12 (IL-12), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

In some embodiments, the concentration of any one of the pro-inflammatory cytokines (e.g., IL-2, IL-6, IL-12, TNF-α, IL-17A, IFN-β, IFN-γ, TGF-β1, and IL-1β) is in the range of 0.01-10000 pg/mL, 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

In some embodiments, the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL, optionally wherein the concentration of IL-2 is 1000 U/mL.

In some embodiments, the concentration of each of the at least three additional pro-inflammatory cytokines is in the range of 100-1000 pg/mL.

In some embodiments, the pro-inflammatory cytokines induce a signaling cascade within the regulatory T cells of the isolated cell population.

In some embodiments, the level of expansion of the isolated cell population is determined relative to a control, optionally wherein the control is a control isolated cell population that is not being cultured in the presence of additional pro-inflammatory cytokines. In some embodiments, the level of expansion of the isolated cell population is increased relative to the control.

In some embodiments, at least 80% of the cells of the isolated cell population are CD25^{+/high}CD4⁺CD127^{-/lo} regulatory T cells. In some embodiments, the method further comprises characterizing the relative abundance of IFN-γ⁺ cells within the cell population after (i). In some embodiments, prior to the culturing of step (i), at least 70% of the cells of the isolated cell population are stable regulatory T cells comprising a hypomethylated TSDR at an endogenous FOXP3 locus and/or at least 80% of the cells of the isolated cell population are FOXP3+. In some embodiments, prior to the culturing of step (i), at least 70% of the cells of the isolated cell population are stable regulatory T cells comprising a hypomethylated TSDR at an endogenous FOXP3 locus. In some embodiments, prior to the culturing of step (i), at least 80% of the cells of the isolated cell population are FOXP3⁺. In some embodiments, prior to the culturing of step (i), at least 70% of the cells of the isolated cell population are stable regulatory T cells comprising a hypomethylated TSDR at an endogenous FOXP3 locus and at least 80% of the cells of the isolated cell population are FOXP3⁺.

In some embodiments, the isolated cell population has undergone one or more cryopreservation freeze-thaw cycles.

In some embodiments, culturing the isolated cell population comprises culturing the isolated cell population in a cell media. In some embodiments, culturing the isolated cell population comprises replacing the cell media with new cell media at regular intervals. The cell media may comprise a buffer (e.g., phosphate-buffered saline (PBS)), bovine serum albumin, human serum albumin, and/or EDTA.

In some embodiments, the isolated cell population is cultured in the presence of one or more agents that activate T cells within the isolated cell population. In some embodiments, the one or more agents that activate T cells are antigen-presenting cells or agents that mimic antigen-presenting cells. The agents that mimic antigen-presenting cells may be three-dimensional beads. In some embodiments, the three-dimensional beads are attached to activation signals. In some embodiments, the agents that mimic antigen-presenting cells are three-dimensional beads that are attached to anti-CD3, anti-CD28, and/or anti-CD137 molecules. In some embodiments, the three-dimensional beads are attached to anti-CD3 molecules. In some embodiments, the three-dimensional beads are attached to anti-CD28 molecules. In some embodiments, the three-dimensional beads are attached to anti-CD137 molecules. In some embodiments, the agents are polystyrene beads coated with a mixture of monoclonal antibodies against the CD3 and CD28 cell surface molecules of human T cells. In some embodiments, the one or more agents that activate T cells are phorbol 12-myristate 13-acetate (PMA) and ionomycin.

In some embodiments, culturing the isolated cell population comprises replacing the cell media with new cell media every 2-5 days, optionally every 3-4 days.

In some embodiments, the isolated cell population is a stable cell population if at least 80% of the regulatory T cells comprise a hypomethylated TSDR and an endogenous FOXP3 locus and/or at least 80% of the cells of the isolated cell population are FOXP3⁺. In some embodiments, the isolated cell population is a stable cell population if at least 80% of the regulatory T cells comprise a hypomethylated TSDR and an endogenous FOXP3 locus. In some embodiments, the isolated cell population is a stable cell population if at least 80% of the cells of the isolated cell population are FOXP3+. In some embodiments, the isolated cell population is a stable cell population if at least 80% of the regulatory T cells comprise a hypomethylated TSDR and an endogenous FOXP3 locus and at least 80% of the cells of the isolated cell population are FOXP3+.

### BRIEF DESCRIPTION OF THE DRAWINGS

FIG. 1 provides a schematic of an exemplary method of a regulatory T cell stability assay of the disclosure.

FIG. 2 provides an exemplary regulatory T cell gating strategy used in the regulatory T cell stability assay of the disclosure that utilizes fluorescence-activated cell sorting (FACS).

FIG. 3 provides graphs showing that regulatory T cells are expanded in the presence of pro-inflammatory cytokines.

FIG. 4 provides graphs showing that regulatory T cells are stable following culture and expansion in the presence of pro-inflammatory cytokines.

FIGS. 5A-5B provide graphs showing the expansion and purity of a cell population comprising regulatory T cells following culture and expansion in the presence of pro-inflammatory cytokines for up to 23 days.

FIG. 6 provides a graph showing the ability of an isolated cell population comprising regulatory T cells to expand when cultured in the presence of IL-2 and TNF-α.

FIGS. 7A-7D provide graphs showing the percentage of CD4⁺ Foxp3⁺ cells and percentage of TSDR demethylation in stable regulatory T cells following culture and expansion in the presence of IL-2 or two different mixtures of pro-inflammatory cytokines for three days. Data for three different runs are shown.

FIGS. 8A-8B is a graph showing the percentage of CD4⁺ Foxp3⁺ cells and percentage of TSDR demethylation in conventional T cells following culture and expansion in the presence of IL-2 or two different mixtures of pro-inflammatory cytokines for three days.

FIG. 9A is a graph showing that FOXP3 expression on Tregs remained stable over a 5-day culture period, even in the presence of the pro-inflammatory Mix-6. In vitro induced Tregs (iTregs) showed a decrease in FOXP3 expression over time after recombinant TGF-β being removed at day 0. FIG. 9B is a graph showing that the demethylation of the TSDR region remained stable and high on Tregs even in the presence of the pro-inflammatory Mix-6; TSDR demethylation remained below 2% for iTregs over time under different culture conditions. FIG. 9C and FIG. 9D are graphs showing that Tregs maintained low IL-2 (FIG. 9C) and IFN-γ (FIG. 9D) over time, even in the presence of proinflammatory Mix-6, while iTregs showed increased expression of both IFN-γ and IL-2 (the Tregs and iTregs were stimulated with PMA and ionomycin for 4 hours and the amount of IL-2 and IFN-γ was detected in the FOXP3+ cells).

FIGS. 10A-10C provide graphs showing the percentage TSDR demethylation in conventional T cells (FIG. 10A), stable regulatory T cells (FIG. 10B), and stable regulatory T cells engineered to express an exogenous TCR (FIG. 10C) following culture and expansion in the presence of IL-2 or two different mixtures of pro-inflammatory cytokines for five days.

FIGS. 11A-11C provide graphs showing the percentage of CD4⁺ Foxp3⁺ cells in conventional T cells (FIG. 11A), stable regulatory T cells (FIG. 11B), and stable regulatory T cells engineered to express an exogenous TCR (FIG. 11C) following culture and expansion in the presence of IL-2 or two different mixtures of pro-inflammatory cytokines for five days. FIGS. 12A-12C provide graphs showing the percentage of IFN-γ⁺ cells in conventional T cells (FIG. 12A), stable regulatory T cells (FIG. 12B), and stable regulatory T cells engineered to express an exogenous TCR (FIG. 12C) following culture and expansion in the presence of IL-2 or two different mixtures of pro-inflammatory cytokines for five days.

### DETAILED DESCRIPTION

The present disclosure provides, in some aspects, methods and compositions for the assessment of regulatory T cell stability over time under inflammatory conditions. In other aspects, the present disclosure provides methods and compositions for expanding regulatory T cells in culture. Cell populations comprising regulatory T cells (e.g., engineered regulatory T cells comprising an antigen-specific T cell receptor (TCR)) are useful for the treatment of autoimmune diseases such as multiple sclerosis (MS), type 1 diabetes (T1D), and inclusion body myositis (IBM). In some embodiments, such cell populations comprise stable (e.g., terminally differentiated) regulatory T cells that are capable of persisting in vivo for extended periods and may provide therapeutic benefit for months or years following a single dose (or, in some instances, multiple doses). These stable regulatory T cells are also, in some embodiments, able to maintain their Treg phenotype in the presence of pro-inflammatory triggers (e.g., pro-inflammatory cytokines).

Suitable characterization of these stable regulatory T cells has proven challenging in the field prior to development efforts presented herein. Following initial production of an isolated cell population comprising regulatory T cells (regulatory T cells are also referred to as “Tregs”), it is often necessary to further evaluate the stability profile of these regulatory T cells under pro-inflammatory conditions (e.g., pro-inflammatory conditions that approximate the in vivo conditions these cells may experience within a human body). For stable regulatory T cells expressing an exogenous TCR, in particular, it is critical that the regulatory T cells maintain a “Treg” phenotype once they are targeted to sites of inflammation in vivo. Any significant population of non-Treg or non-stable Treg (peripheral Treg) that are able to convert into conventional T cells could exacerbate, rather than suppress, an immune response. The assay presented herein is an ex vivo assay that assesses whether Tregs will maintain their stable Treg phenotype once exposed to inflammatory conditions, and thereby, whether the Treg are safe for administration to a subject.

Accordingly, useful methods for evaluating the stability profiles of these isolated cell population comprising regulatory T cells are provided by the disclosure. For example, in some embodiments, provided herein is a method of evaluating the stability profile of an isolated cell population comprising regulatory T cells, wherein the method comprises culturing the isolated cell population in the presence of one or more (e.g., two or more) pro-inflammatory cytokines (e.g., 2, 3, 4, 5, 6, 7, 8, or 9 pro-inflammatory cytokines) for a period of time, characterizing one or more markers of stability (e.g., FOXP3 expression or the methylation status of a regulatory T cell-specific demethylation region (TSDR) at an endogenous FOXP3 locus) of the isolated cell population following the period of time, and evaluating the stability profile of the isolated cell population based on the characterizing of the one or more markers of stability.

In some embodiments, the method further comprises determining the levels of pro-inflammatory cytokines produced by the cells (e.g., regulatory T cells) of the isolated cell population. In some embodiments, the method further comprises determining the levels of intracellular IFN-γ produced by the cells (e.g., regulatory T cells) of the isolated cell population. Specifically, the method, in some embodiments, involves a determination of the relative abundance of IFN-γ+cells within the isolated cell population after exposure to inflammatory cytokines. In some embodiments, the method further comprises determining the levels of intracellular IL-17 produced by the cells (e.g., regulatory T cells) of the isolated cell population. In some embodiments, the method involves a determination of the relative abundance of IL-17⁺ cells within the isolated cell population after exposure to inflammatory cytokines. In some embodiments, the method further comprises determining the levels of intracellular TNF-α produced by the cells (e.g., regulatory T cells) of the isolated cell population. In some embodiments, the method involves a determination of the relative abundance of TNF-α⁺ cells within the isolated cell population after exposure to inflammatory cytokines. In some embodiments, the method further comprises determining the levels of IL-2 produced by the cells (e.g., regulatory T cells) of the isolated cell population. Accordingly, in some embodiments, involves a determination of the relative abundance of IL-2 producing cells within the isolated cell population after exposure to inflammatory cytokines. This characterization is intended to determine whether the regulatory T cells of the isolated cell population adopt effector cell function in response to inflammatory stimuli. Furthermore, large-scale production of these regulatory T cells necessitates, in some embodiments, efficient methods of culturing and expanding regulatory T cells. Surprisingly, the data provided herein shows that culturing regulatory T cells in the presence of certain pro-inflammatory cytokines and specific combinations of pro-inflammatory cytokines facilitate high levels of regulatory T cell expansion.

### Regulatory T Cells

The present disclosure provides methods of evaluating the stability profile, expanding, and/or culturing stable regulatory T cells and compositions comprising such stable regulatory T cells. A regulatory T cell, also referred to as a “Treg”, is a T cell that modulates the immune system. Regulatory T cells are immunosuppressive and generally suppress or downregulate induction and proliferation of effector T cells. Regulatory T cells are thought to be derived from the same lineage as naïve CD4⁺ cells and express the biomarkers CD4, CD25, and FOXP3. In some embodiments, the regulatory T cells (e.g., stable regulatory T cells) comprise an exogenous human T cell receptor (TCR) that binds specifically to target peptide. This binding occurs when the target peptide is complexed with a major histocompatibility complex (MHC) (e.g., MHC Class I or MHC Class II).

A stable, or thymic, regulatory T cell is a regulatory T cell that comprises a hypomethylated TSDR at an endogenous FOXP3 locus and expresses CD4, CD25, and FOXP3. A regulatory T cell is considered “stable” if it is terminally differentiated, i.e., it has lost its ability to change its cell fate. In thymic regulatory T cell development, the genome organizer SATB1 (special AT-rich sequence-binding protein) binds to specific genomic sites from the CD4⁺ CD8⁺ thymocyte stage to open up the chromatin and activate super-enhancers associated with many regulatory T cell signature genes such as FOXP3, IL2RA, (CD25), CTLA4, IKZF2 (HELIOS), and IFZF4 (EOS). SATB1 and MLL4 (myeloid/lymphoid or mixed-lineage leukemia 4), an enzyme involved in enhancer priming, commonly occupy the newly identified conserved enhancer region, designated conserved noncoding sequence 0 (CNS0), at the FOXP3 locus, with subsequent activation of the enhancers at CNS3 and CNS2, and then the promoter. This results in stable hypomethylation and expression of FOXP3 and other regulator T cell-associated genes, thereby resulting in a stable regulatory T cell phenotype. Epigenetic mechanisms, in particular, DNA methylation/demethylation, which is heritable through cell divisions, play an essential role for stable maintenance of regulatory T cell-specific gene expression. Hypomethylation of the TSDR, which is an evolutionary conserved CpG-rich regulatory element of the FOXP3 gene, is associated with expression of FOXP3. A TSDR of an endogenous FOXP3 locus is hypomethylated when the methyl group from one or more methylated cytosines in the TSDR have been removed to replace the methylated cytosine(s) with cytosine. In some embodiments, measurement of the methylation status of the TSDR of a FOXP3 locus is as described in Kressler et. al. “Targeted De-Methylation of the FOXP3-TSDR Is Sufficient to Induce Physiological FOXP3 Expression but Not a Functional Regulatory T Phenotype” Frontiers in Immunology, 7 Jan. 2021.; or Schreiber, et. al. “The Regulatory T-Specific Demethylated Region Stabilizes Foxp3 Expression Independently of NF-κB Signaling” PLOS One, Feb. 5, 2014. Genomic DNA is isolated and bisulfite conversion is performed. This converts, in some embodiments, all the unmethylated cysteines to uracils. The TSDR region can be amplified using primers that amplify the bisulfite-converted DNA, independent of methylation status. Two competing probes detect the abundance of methylated or unmethylated CpG, with the total and/or relative abundance being determined by ddPCR or pyrosequencing. Alternatively, next-generation sequencing (NGS) can be performed on the bisulfite-converted DNA to determine the sequence of the bisulfite-converted DNA at the TSDR locus.

In some embodiments, a stable regulatory T cell retains one or more markers of stability in the presence of pro-inflammatory conditions (e.g., in presence of one or more pro-inflammatory cytokines). In some embodiments, a stable regulatory T cell expresses FOXP3 in the presence of pro-inflammatory conditions (e.g., in presence of one or more pro-inflammatory cytokines). In some embodiments, a stable regulatory T cell expresses CD4, CD25, and FOXP3 in the presence of pro-inflammatory conditions (e.g., in presence of one or more pro-inflammatory cytokines). In some embodiments, a stable regulatory T cell comprises a hypomethylated TSDR at an endogenous FOXP3 locus in the presence of pro-inflammatory conditions (e.g., in presence of one or more pro-inflammatory cytokines). In some embodiments, a stable regulatory T cell comprises a hypomethylated TSDR at an endogenous FOXP3 locus and expresses CD4, CD25, and FOXP3 in the presence of pro-inflammatory conditions (e.g., in presence of one or more pro-inflammatory cytokines). In some embodiments, a stable regulatory T cell expresses CD4, CD25, and FOXP3 in the presence of pro-inflammatory conditions for at least 3, 4, 5, 6, 7, 8, 9, 10, 11, 12, 13, 14, 15, 20, 21, 22, 23, 24, or 25 days. In some embodiments, a stable regulatory T cell comprises a hypomethylated TSDR at an endogenous FOXP3 locus in the presence of pro-inflammatory conditions for at least 3, 4, 5, 6, 7, 8, 9, 10, 11, 12, 13, 14, 15, 20, 21, 22, 23, 24, or 25 days. In some embodiments, a stable regulatory T cell comprises a hypomethylated TSDR at an endogenous FOXP3 locus and expresses CD4, CD25, and FOXP3 in the presence of pro-inflammatory conditions for at least 3, 4, 5, 6, 7, 8, 9, 10, 11, 12, 13, 14, 15, 20, 21, 22, 23, 24, or 25 days.

Furthermore, in some embodiments, a stable regulatory T cell has a low level of intracellular IFNγ expression. In some embodiments, a stable regulatory T cell maintains a low level of intracellular IFNγ expression in the presence of pro-inflammatory conditions for at least 3, 4, 5, 6, 7, 8, 9, 10, 11, 12, 13, 14, 15, 20, 21, 22, 23, 24, or 25 days. In some embodiments, a stable regulatory T cell does not adopt or have effector cell function.

The one or more markers of stability of a regulatory T cell are one or more biomarkers that are representative of a stable regulatory T cell. In some embodiments, a marker of stability of a regulatory T cell is a hypomethylated TSDR at an endogenous FOXP3 locus. In some embodiments, a marker of stability of a regulatory T cell is CD4, CD25, or FOXP3. In some embodiments, a marker of stability (e.g., CD4, CD25, or FOXP3) is characterized by determining expression or activity of said marker. In some embodiments, a regulatory T cell retains a marker of stability if the regulatory T cell maintains a hypomethylated TSDR at an endogenous FOXP3 locus after a period of time. In some embodiments, a regulatory T cell retains a marker of stability if the regulatory T cell maintains expression of CD4, CD25, and/or FOXP3 after a period of time. In some embodiments, a regulatory T cell retains a marker of stability if the regulatory T cell maintains high levels of expression of CD4, CD25, and/or FOXP3 after a period of time.

A stable regulatory T cell further exhibits one or more of the following functions: (i) cytokine secretion activity (e.g., secretion of IL-10, IL-4, IL-6, IL-11, and/or IL-13) when activated (e.g., activated by anti-CD3 and anti-CD28 antibodies); (ii) expression of activation markers associated with regulatory T cells (e.g., expression of CD69, 4-1BB, CD25, CD71, and/or CTLA-4) when activated (e.g., activated by anti-CD3 and anti-CD28 antibodies); and/or (iii) suppression activity (e.g., the ability of the stable regulatory T cell to suppress the activation of conventional T cells having specificity towards a shared target peptide) when activated (e.g., activated by anti-CD3 and anti-CD28 antibodies).

A stable regulatory T cell that expresses an exogenous TCR that binds specifically to a target peptide complexed with an MHC further exhibits one or more of the following functions: (i) cytokine secretion activity (e.g., secretion of IL-10, IL-4, IL-6, IL-11, and/or IL-13) when the TCR is contacted with its target peptide; (ii) expression of activation markers associated with regulatory T cells (e.g., expression of CD69, 4-1BB, CD25, CD71, and/or CTLA-4) when the TCR is contacted with its target peptide; and/or (iii) suppression activity (e.g., the ability of the stable regulatory T cell to suppress the activation of conventional T cells having specificity towards a shared target peptide) when the TCR is contacted with the target peptide complexed with an MHC.

Thus, regulatory T cells of isolated cell populations provided herein may comprise a hypomethylated TSDR at an endogenous FOXP3 locus, expression of CD4, CD25, and FOXP3, and/or exhibit cytokine secretion, activation, and/or suppression activity. In some embodiments, regulatory T cells of isolated cell populations comprise a hypomethylated TSDR at an endogenous FOXP3 locus. In some embodiments, regulatory T cells of isolated cell populations express CD4, CD25, and FOXP3. In some embodiments, regulatory T cells of isolated cell populations exhibit cytokine secretion activity. In some embodiments, regulatory T cells of isolated cell populations exhibit activation activity. In some embodiments, regulatory T cells of isolated cell populations exhibit suppression activity.

In some embodiments, a regulatory T cell (e.g., a stable regulatory T cell) is CD25⁺. In some embodiments, a regulatory T cell (e.g., a stable regulatory T cell) is CD25^{+/high}. As is used herein, “CD25^{+/high}” means that the cell is either CD25⁺ or CD25^{high}. A CD25⁺ cell is a cell that expresses a detectable level of CD25 (e.g., detectable by FACS). A CD25^{high} cell is a cell that expresses CD25 above a threshold or control level. For example, in the context of an isolated cell population that comprises CD25⁺ cells, a subpopulation of those cells may express higher levels of CD25 relative to another subpopulation of CD25⁺ cells in that population (or relative to all the other cells CD25⁺ cells in the population). The former subpopulation would be considered to be CD25^{high}, while the latter subpopulation would be considered only CD25⁺ (not CD25^{high}).

In some embodiments, a regulatory T cell (e.g., a stable regulatory T cell) is CD4⁺.

In some embodiments, a regulatory T cell (e.g., a stable regulatory T cell) is CD127^{-/lo}. As is used herein, “CD127^{-/lo}” means that the cell is either CD127⁻ or CD127^{lo}. A CD127⁻ cell is a cell that does not express CD127, does not express a detectable level of CD127 (e.g., by FACS), or expresses CD127 below a threshold or control level. For example, in the context of an isolated cell population that comprises at least some proportion of CD127⁺ cells (e.g., detectable by FACS), a subpopulation of those cells may express lower levels of CD127 relative to another subpopulation of CD127⁺ cells in that population (or relative to all the other cells CD127+ cells in the population). The former subpopulation would be considered to be CD127^{-/lo}, while the latter subpopulation would be considered only CD127^{lo} (not CD127⁻).

In some embodiments, a regulatory T cell (e.g., a stable regulatory T cell) is CD45RA⁺.

In some embodiments, a regulatory T cell (e.g., a stable regulatory T cell) is FOXP3⁺.

In some embodiments, a regulatory T cell (e.g., a stable regulatory T cell) is a CD25^{+/high}CD4⁺CD127^{-/lo} cell. That is, the regulatory T cell expresses, or expresses a high level of CD25, expresses CD4, and does not express, or expresses a low level of, CD127. In some embodiments, a regulatory T cell does not express CD127. In some embodiments, a regulatory T cell (e.g., a stable regulatory T cell) is a CD25^{+/high}CD4⁺CD127^{-/10/}CD45RA⁺ cell. Thus, the regulatory T cells expresses, or expresses a high level of CD25, expresses CD4 and CD45RA, and does not express, or expresses a low level of, CD127. In some embodiments, a regulatory T cell expresses FOXP3. In some embodiments, a regulatory T cell is a CD25^{+/high}CD4+CD127^{-/lo}FOXP3⁺ cell. Thus, the regulatory T cells expresses, or expresses a high level of CD25, expresses CD4 and FOXP3, and does not express, or expresses a low level of, CD127. In some embodiments, a regulatory T cell is a CD25^{+/high}CD4⁺/CD45RA⁺/CD127^{-/lo} FOXP3⁺cell. Thus, the regulatory T cells expresses, or expresses a high level of CD25, expresses CD4, CD45RA, and FOXP3, and do not express, or expresses a low level of, CD127.

A cell “expresses” a biomarker (e.g., CD4, CD45RA, CD25, and/or FOXP3) if the biomarker can be detected using a conventional protein expression assay, such as an antibody detection assay. An antibody detection assay in this context involves the detection (e.g., using fluorescence activated cell sorting (FACS) or Western blot) of an antibody that binds to the biomarker of interest (e.g., CD4, CD45RA,CD25, or FOXP3) when the biomarker is present within a cell or on the surface of a cell. A cell “does not express” a biomarker (e.g., CD127) if the expression of the biomarker cannot be detected using a conventional protein assay. A cell “expresses a low level” of a biomarker (e.g., CD127), if expression of that biomarker is lower than (e.g., at least 50%, at least 60%, at least 70%, at least 80%, or at least 90% lower than) a control level. In some embodiments, the control level of a biomarker in a regulatory T cell is the expression level of the biomarker (e.g., CD127) in a conventional T cell or a CD8⁺ T cell. For example, a regulatory T cell expresses a low level of CD127 if its expression of CD127 is lower than (e.g., at least 50%, at least 60%, at least 70%, at least 80%, or at least 90% lower than) expression of CD127 in a conventional T cell.

In some embodiments, a stable regulatory T cell of an isolated cell population provided herein maintains a hypomethylated TSDR at an endogenous FOXP3 locus (e.g., in the presence of one or more pro-inflammatory cytokines) over time (i.e., the TSDR at the endogenous FOXP3 locus of viable cells (e.g., in culture, in a cryoprotective agent, and/or in vivo) is hypomethylated for a certain measurable period of time). For example, a stable regulatory T cell of an isolated cell population may maintain a hypomethylated TSDR at an endogenous FOXP3 locus (e.g., in the presence of one or more pro-inflammatory cytokines) for at least 1, 2, 3, 4, 5, 6, 7, 8, 9, 10, 11, 12, 13, 14, 15, 16, 17, 18, 19, or 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus (e.g., in the presence of one or more pro-inflammatory cytokines) for more than 5 days, more than 10 days, more than 15 days, or more than 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus (e.g., in the presence of one or more pro-inflammatory cytokines) for 1-20 days, 1-10 days, 1-5 days, 5 -30 days, 5-20 days, 10-40days, or 25-50 days (e.g., in culture, in a cryoprotective agent, and/or in vivo after being obtained from a subject or following transduction with a nucleic acid expressing a TCR).

A regulatory T cell of an isolated cell population of the disclosure, in some embodiments, is an autologous cell. The term autologous in this context refers to cells that have been obtained from the same subject to which they are subsequently administered (e.g., following engineering to introduce an exogenous TCR into the cells). For example, a population of cells may be obtained from a subject, subjected to the methods described herein, and then administered to the same subject (from which the population of cells was originally obtained) to treat an autoimmune disease. In such embodiments, the population of cells administered to the subject comprise autologous regulatory T cells.

A regulatory T cell of the disclosure, in some embodiments, is an allogenic cell. The term allogenic in this context refers to cells that have been obtained from one subject and then administered to another subject. For example, a population of cells may be obtained from a subject, subjected to the methods described herein, and then administered to another subject in order to treat an autoimmune disease.

In some embodiments, regulatory T cells (e.g., a biological sample comprising regulatory T cells) are obtained from a subject diagnosed with, or suspected of having, an autoimmune disease. The autoimmune disease may be selected from Multiple Sclerosis (e.g., progressive Multiple Sclerosis), Type 1 Diabetes, and Inclusion Body Myositis. In some embodiments, the autoimmune disease is progressive Multiple Sclerosis (e.g., progressive Multiple Sclerosis). In some embodiments, regulatory T cells are obtained from a subject diagnosed with or suspected of having Multiple Sclerosis (e.g., progressive Multiple Sclerosis). In other embodiments, the autoimmune disease is Type 1 Diabetes. In some embodiments, regulatory T cells are obtained from a subject diagnosed with or suspected of having Type 1 Diabetes. In yet other embodiments, the autoimmune disease is Inclusion Body Myositis. In some embodiments, regulatory T cells are obtained from a subject diagnosed with or suspected of having Inclusion Body Myositis.

### Methods of Evaluating the Stability Profile of Regulatory T Cells

Evaluation of the stability profile of regulatory T cells is a valuable tool in characterizing the quality of an isolated cell population comprising regulatory T cells, particularly if the isolated cell population is intended to be used to therapeutic purposes. The stability profile of regulatory T cells may be evaluated by subjecting the regulatory T cells to pro-inflammatory conditions (e.g., such as those that might be experienced within a patient). The stability profile of regulatory T cells may include an evaluation of the methylation status of a TSDR at an endogenous FOXP3 locus, the expression and/or activity of CD4, CD25, and/or FOXP3. In some embodiments, the stability profile of an isolated cell population comprising regulatory T cells is evaluated by (i) culturing the isolated cell population in the presence of two or more pro-inflammatory cytokines for a period of time; (ii) characterizing one or more markers of stability of the isolated cell population following the period of time; and (iii) evaluating the stability profile of the isolated cell population based on the characterizing of the one or more markers of stability.

In some embodiments, a pro-inflammatory cytokine is Interleukin-2 (IL-2), Interleukin-6 (IL-6), Interleukin-12 (IL-12) (IL-12p70), Tumor necrosis factor alpha (TNF-α), Interleukin-17 (IL-17A), Interferon gamma (IFN-γ), Interferon beta (IFN-β), Transforming growth factor beta 1 (TGF-β1), Interleukin-1 beta (IL-1β), Interleukin-22 (IL-22), Granulocyte-macrophage colony-stimulating factor (GM-CSF) or Interleukin-23 (IL-23). In some embodiments, a method of evaluating the stability profile of a regulatory T cell comprises culturing an isolated cell population comprising the regulatory T cell in the presence of at least two, at least three, at least four, at least five, at least six, at least seven, at least eight, or at least nine pro-inflammatory cytokines.

In some embodiments, a method of evaluating the stability profile of a regulatory T cell comprises culturing an isolated cell population comprising the regulatory T cell in the presence of IL-2 and IL-6. In some embodiments, a method of evaluating the stability profile of a regulatory T cell comprises culturing an isolated cell population comprising the regulatory T cell in the presence of IL-2 and IL-12. In some embodiments, a method of evaluating the stability profile of a regulatory T cell comprises culturing an isolated cell population comprising the regulatory T cell in the presence of IL-2 and TNF-α. In some embodiments, a method of evaluating the stability profile of a regulatory T cell comprises culturing an isolated cell population comprising the regulatory T cell in the presence of IL-2 and IL-17A. In some embodiments, a method of evaluating the stability profile of a regulatory T cell comprises culturing an isolated cell population comprising the regulatory T cell in the presence of IL-2 and IFN-β. In some embodiments, a method of evaluating the stability profile of a regulatory T cell comprises culturing an isolated cell population comprising the regulatory T cell in the presence of IL-2 and IFN-γ. In some embodiments, a method of evaluating the stability profile of a regulatory T cell comprises culturing an isolated cell population comprising the regulatory T cell in the presence of IL-2 and TGF-β1. In some embodiments, a method of evaluating the stability profile of a regulatory T cell comprises culturing an isolated cell population comprising the regulatory T cell in the presence of IL-2 and IL-1β.

In some embodiments, a method of evaluating the stability profile of a regulatory T cell comprises culturing an isolated cell population comprising the regulatory T cell in the presence of IL-2, IL-6, and at least one (e.g., 1, 2, 3, 4, 5, 6, or 7) additional pro-inflammatory cytokine selected from: IL-12, TNF-α, IL-17A, IFN-β, IFN-γ, TGF-β1, and IL-1β. In some embodiments, a method of evaluating the stability profile of a regulatory T cell comprises culturing an isolated cell population comprising the regulatory T cell in the presence of IL-2, IL-12, and at least one (e.g., 1, 2, 3, 4, 5, 6, or 7) additional pro-inflammatory cytokine selected from: IL-6, TNF-α, IL-17A, IFN-β, IFN-γ, TGF-β1, and IL-1β. In some embodiments, a method of evaluating the stability profile of a regulatory T cell comprises culturing an isolated cell population comprising the regulatory T cell in the presence of IL-2, TNF-α, and at least one (e.g., 1, 2, 3, 4, 5, 6, or 7) additional pro-inflammatory cytokine selected from: IL-6, IL-12, IL-17A, IFN-β, IFN-γ, TGF-β1, and IL-1β. In some embodiments, a method of evaluating the stability profile of a regulatory T cell comprises culturing an isolated cell population comprising the regulatory T cell in the presence of IL-2, IL-17A, and at least one (e.g., 1, 2, 3, 4, 5, 6, or 7) additional pro-inflammatory cytokine selected from: IL-6, IL-12, TNF-α, IFN-β, IFN-γ, TGF-β1, and IL-1β. In some embodiments, a method of evaluating the stability profile of a regulatory T cell comprises culturing an isolated cell population comprising the regulatory T cell in the presence of IL-2, IFN-β, and at least one (e.g., 1, 2, 3, 4, 5, 6, or 7) additional pro-inflammatory cytokine selected from: IL-6, IL-12, TNF-α, IL-17A, IFN-γ, TGF-β1, and IL-1β. In some embodiments, a method of evaluating the stability profile of a regulatory T cell comprises culturing an isolated cell population comprising the regulatory T cell in the presence of IL-2, IFN-γ, and at least one (e.g., 1, 2, 3, 4, 5, 6, or 7) additional pro-inflammatory cytokine selected from: IL-6, IL-12, TNF-α, IL-17A, IFN-β, TGF-β1, and IL-1β. In some embodiments, a method of evaluating the stability profile of a regulatory T cell comprises culturing an isolated cell population comprising the regulatory T cell in the presence of IL-2, TGF-β1, and at least one (e.g., 1, 2, 3, 4, 5, 6, or 7) additional pro-inflammatory cytokine selected from: IL-6, IL-12, TNF-α, IL-17A, IFN-β, IFN-γ, and IL-1β. In some embodiments, a method of evaluating the stability profile of a regulatory T cell comprises culturing an isolated cell population comprising the regulatory T cell in the presence of IL-2, IL-1β, and at least one (e.g., 1, 2, 3, 4, 5, 6, or 7) additional pro-inflammatory cytokine selected from: IL-6, IL-12, TNF-α, IL-17A, IFN-β, IFN-γ, and TGF-β1.

In some embodiments, a method of evaluating the stability profile of a regulatory T cell comprises culturing an isolated cell population comprising the regulatory T cell in the presence of IL-2, IL-6, IL-12, TNF-α, IL-17A, IFN-β, IFN-γ, TGF-β1, and IL-1β.

In some embodiments, a method of evaluating the stability profile of a regulatory T cell comprises culturing an isolated cell population comprising the regulatory T cell in the presence of IL-2, IL-1β, IL-6, IL-12, IL-17A, IFN-γ, TNF-α, IFN-β, and TGF-β1.

In some embodiments, a method of evaluating the stability profile of a regulatory T cell comprises culturing an isolated cell population comprising the regulatory T cell in the presence of IL-2, IL-1β, IL-6, IL-12, IL-17A, IFN-γ, and TNF-α.

The concentration of any one of the pro-inflammatory cytokines (e.g., IL-2, IL-1β, IL-6, IL-12, TNF-α, IL-17A, IFN-β, IFN-γ, and TGF-β1) may be in the range of 0.01-10000 pg/mL, 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL. In some embodiments, the concentration of any one of the pro-inflammatory cytokines is about 500, about 1000, about 1500, or about 2000 pg/ml. In some embodiments, the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL. In some embodiments, the concentration of IL-2 is about 500, about 1000, about 1500, or about 2000 U/ml.

In some embodiments, the isolated cell population is cultured in the presence of two or more pro-inflammatory cytokines under activating conditions. In some embodiments, the isolated cell population is cultured in the presence of two or more pro-inflammatory cytokines and anti-CD3 and anti-CD28 antibodies. In some embodiments, the anti-CD3 and anti-CD8 antibodies are attached to a magnetic bead, such as CD3/CD28 Dynabeads™. CD3/CD28 Dynabeads™ are uniform 4.5 μm, super-paramagnetic polystyrene beads coated with a mixture of monoclonal antibodies against the CD3 and CD28 cell surface molecules of human T cells. In some embodiments, the anti-CD3 and anti-CD8 antibodies are suspended in a matrix, such as Transact™. In some embodiments, when the regulatory T cells express an exogenous TCR, the isolated cell population is cultured in the presence of two or more pro-inflammatory cytokines and a TCR ligand, e.g., a peptide MHC complex. In some embodiments, the peptide MHC complex is presented by an antigen presenting cell. In some embodiments, the peptide-MHC complex is presented by a multimer reagent, e.g., a tetramer or dextramer. In some embodiments, the isolated cell population is cultured in the presence of one or more pro-inflammatory cytokines, phorbol 12-myristate 13-acetate (PMA) and ionomycin to activate intracellular signaling pathways. In such embodiments, PMA and ionomycin function to activate the T cells of the isolated cell population through an activation of protein kinase C (PKC) and nuclear factor of activated T-cell (NFAT) signaling pathways. In some embodiments, the isolated cell population is activated after exposing the isolated cell population to pro-inflammatory conditions (e.g., culturing in the presence of one or more pro-inflammatory cytokines for a period of time). In some embodiments, a step of activating the isolated cell population after exposure to pro-inflammatory conditions is performed prior to (e.g., immediately prior to) characterization of the isolated cell population (e.g., characterization of one or more markers of stability of regulatory T cells). In some embodiments, the isolated cell population is activated after exposing the isolated cell population to pro-inflammatory conditions using a combination of PMA and ionomycin.

In some embodiments, a method of evaluating the stability profile of a regulatory T cell comprises culturing an isolated cell population comprising the regulatory T cell in the presence of pro-inflammatory cytokines for a period of time. A period of time in relation to the length of time for culturing an isolated population comprising regulatory T cells may be as short as 1, 2, or 3 days, or as long as 2 or 3 months. In some embodiments, a period of time is about or at least 3, at least 4, at least 5 at least 6, at least 7, at least 8, at least 9, at least 10, at least 11, at least 12, at least 15, at least 20, at least 23, at least 25, or at least 30 days. In some embodiments, a period of time is 1-30 days, 2-30 days, 2-20 days, 2-10 days, 5-20 days, 5-10 days, 3-25 days, 5-25 days, 5-20 days, 10-20 days, 10-25 days, 5-10 days, 10-30 days, 10-25 days, 10-20 days, 15-30 days, 20-30 days, 10-60 days, 10-50 days, 10-40 days, or 20-40 days. In some embodiments, a period of time is 1, 2, 3, 4, 5, 6, 7, 8, 9, 10, 11, 12, 13, 14, 20, 23, 25, or 30 days. In some embodiments, a period of time is 1-6 weeks, 1-5 weeks, 1-4 weeks, 1-3 weeks, 1-2 weeks, or 2-4 weeks.

Cells may be plated in a cell culture dish or a multiwell plate (e.g., 96-well plate) at a concentration of at least 10² cells/mL, at least 10³ cells/mL, at least 10⁴ cells/mL, at least 10⁵ cells/mL, at least 10⁶ cells/mL, at least 10⁷ cells/mL, at least 10⁸ cells/mL, at least 10⁹ cells/mL, or at least 10¹⁰ cells/mL. In some embodiments, the concentration of cells is 10² to 10¹⁰ cells/mL, 10² to 10³ cells/mL, 10² to 10⁴ cells/mL, 10² to 10⁵ cells/mL, 10² to 10⁶ cells/mL, 10² to 10⁷ cells/mL, 10² to 10⁸ cells/mL, 10² to 10⁹ cells/mL, or 10² to 10¹⁰ cells/mL.

The one of more markers of stability may be characterized multiple time during the period of time. In some embodiments, the one or more markers of stability are characterized at regular intervals throughout the period of time. For example, in some embodiments, the one or more markers of stability are characterized every 2-5 days throughout the period of time. In some embodiments, the one or more markers of stability are characterized every day, every second day, every third day, every fourth day, every fifth day, every six days, or once per week throughout the period of time. In some embodiments, the one or more markers of stability are characterized prior to initiation of the period of time (e.g., prior to culturing of the isolated cell population. In some embodiments, the one or more markers of stability are characterized only at the end of the period of time.

Characterizing the one or more markers of stability may involve, in some embodiments, determining the expression and/or activity of the one or more markers of stability. For example, characterizing CD4 may involve determining the expression and/or activity of CD4. In some embodiments, characterizing CD25 may involve determining the expression and/or activity of CD25. In some embodiments, characterizing FOXP3 may involve determining the expression and/or activity of FOXP3.

Determining expression and/or activity of one or more markers of stability may be performed using biochemical techniques. As described herein, biochemical techniques refer to methods used in a laboratory to study biochemical processes, identify the chemicals involved in a biochemical reaction, measure the rate of a biochemical reaction, or determine the structure of a protein or other molecule. In some embodiments, the biochemical techniques may be used to determine expression and/or activity on cytokines. For example, antibody-based assays, FACS-based assays, bead-based immunoassays or methods of measuring ribonucleic acid (RNA) or deoxyribonucleic acid (DNA).

Examples of antibody-based assays include, but are not limited to immunohistochemistry, immunohistochemical staining and enzyme-linked immunosorbance assay (ELISA). ELISA is a quantitative assay that may be used to detect secreted cytokines at the protein level. For a description of commonly used antibody-based techniques see Amsen D, et al. Approaches to determine expression of inflammatory cytokines. Methods Mol Biol. 2009; 511:107-42. doi: 10.1007/978-1-59745-447-6_5. PMID: 19347295; PMCID: PMC2698024. Examples of FACS-based assays include but are not limited to flow cytometry (FCM). FCM is a method of detecting intracellular cytokines using specific fluorescence-labeled antibodies. The common steps of this assay include cell collection, fixation, permeabilization, blocking, intracellular staining and analysis by FCM. For a description of FCM see Qiu, Jian-Ge et al. “Cytokine detection by flow cytometry.” Methods in molecular biology (Clifton, N.J.) vol. 1172 (2014): 235-42. doi: 10.1007/978-1-4939-0928-5_21. Examples of bead-based immunoassays include but are not limited to cytokine bead array. Cytokine bead arrays may be used to detect a panel of cytokines in a multiplex fashion using small sample volumes. For a description of commonly used cytokine bead array see Amsen D, et al. Approaches to determine expression of inflammatory cytokines. Methods Mol Biol. 2009;511:107-42. doi: 10.1007/978-1-59745-447-6_5. PMID: 19347295; PMCID: PMC2698024. There are also commercially available kits, such as LEGENDPLEX™ (BioLegend, Inc.). Examples of measuring RNA or DNA include, but are not limited to, real-time quantitative polymerase chain reaction (Q-PCR), Real-Time PCR. Q-PCR is a highly sensitive, quantitative method that involves the measurement of cytokine messenger RNA (mRNA) transcript abundance. Q-PCR may be used to detect many different cytokines from relatively small sample amounts. For a description of commonly used methods of measuring RNA or DNA see Amsen D, et al. Approaches to determine expression of inflammatory cytokines. Methods Mol Biol. 2009; 511:107-42. doi: 10.1007/978-1-59745-447-6_5. PMID: 19347295; PMCID: PMC2698024.

Characterizing the methylation status of a TSDR at an endogenous FOXP3 locus may be performed using an assay as described in Kressler et. al. “Targeted De-Methylation of the FOXP3-TSDR Is Sufficient to Induce Physiological FOXP3 Expression but Not a Functional Regulatory T Phenotype” Frontiers in Immunology, 7 Jan. 2021.; or Schreiber, et. al. “The Regulatory T-Specific Demethylated Region Stabilizes Foxp3 Expression Independently of NF-κB Signaling” PLOS One, Feb. 5, 2014.

In some embodiments, the method further comprises determining the levels of intracellular IFN-γ produced by the cells (e.g., regulatory T cells) of an isolated cell population. In some embodiments, determining the levels of intracellular IFN-γ involves a determination of the relative abundance of IFN-γ⁺ cells within an isolated cell population after exposure to inflammatory cytokines. In some embodiments, the levels of intracellular IFN-γ are determined by staining the cells of the isolated cell population for the presence of intracellular IFN-γ (e.g., using an anti-IFN-γ antibody). In some embodiments, the levels of intracellular IFN-γ are determined using a cell sorting technique or an ELISA assay. In some embodiments, the levels of intracellular IFN-γ are determined using any known IFN-γ release assay or any other method known to a person of ordinary skill directed to determination of IFN-γ production.

In some embodiments, the method further comprises determining the levels of intracellular IL-17 produced by the cells (e.g., regulatory T cells) of an isolated cell population. In some embodiments, determining the levels of intracellular IL-17 involves a determination of the relative abundance of IL-17⁺ cells within an isolated cell population after exposure to inflammatory cytokines. In some embodiments, the levels of intracellular IL-17 are determined by staining the cells of the isolated cell population for the presence of intracellular IL-17 (e.g., using an anti-IL-17 antibody). In some embodiments, the levels of intracellular IL-17 are determined using a cell sorting technique or an ELISA assay. In some embodiments, the levels of intracellular IL-17 are determined using method known to a person of ordinary skill directed to determination of IL-17 production.

In some embodiments, the method further comprises determining the levels of intracellular TNF-α produced by the cells (e.g., regulatory T cells) of an isolated cell population. In some embodiments, determining the levels of intracellular TNF-α involves a determination of the relative abundance of TNF-α⁺ cells within an isolated cell population after exposure to inflammatory cytokines. In some embodiments, the levels of intracellular TNF-α are determined by staining the cells of the isolated cell population for the presence of intracellular TNF-α (e.g., using an anti-TNF-α antibody). In some embodiments, the levels of intracellular TNF-α are determined using a cell sorting technique or an ELISA assay. In some embodiments, the levels of intracellular TNF-α are determined using any known TNF-αrelease assay or any other method known to a person of ordinary skill directed to determination of TNF-α production.

In some embodiments, the method further comprises determining the levels of IL-2 produced by the cells (e.g., regulatory T cells) of an isolated cell population. In some embodiments, determining the levels of IL-2 involves a determination of the relative abundance of IL-2 producing cells within an isolated cell population after exposure to inflammatory cytokines.

An isolated cell population comprising regulatory T cells is typically a cell population that is removed from a subject (e.g., a human subject). Thus, it is considered “isolated” from the subject. A population of cells may be isolated (e.g., obtained from) a subject, or from a biological sample obtained from the subject, for example, using any known cell collection method, such as apheresis. An isolated cell population of the disclosure may be subjected to the methods described herein to produce an isolated cell population a higher number of regulatory T cells (e.g., stable regulatory T cells) relative to a population of cells obtained directly from a subject, or from a biological sample obtained from the subject, by apheresis, for example. A subject refers to an individual organism, for example, an individual human. In some embodiments, the subject is a human subject, such as a male subject or a female subject. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a research animal. In some embodiments, the subject is genetically engineered, e.g., a genetically engineered non-human subject. The subject may be male or female.

In some embodiments, an isolated cell population comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ regulatory T cells. In some embodiments, an isolated cell population comprises 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁵ to 1×10⁹, 1×10⁶ to 1×10⁸, 1×10⁷ to 1×10¹⁰, or 1×10⁴ to 1×10⁶ regulatory T cells. In some embodiments, an isolated cell population comprises 1×10⁶ to 1×10¹⁰ regulatory T cells.

An isolated cell population comprising regulatory T cells may comprise non-regulatory T cells (e.g., conventional T cells). Non-regulatory T cells may be NK T cells, B cells, CD8⁺ T cells, neutrophils, eosinophils, CD14⁺ cells, or conventional (CD4⁺) T cells that are derived from peripheral blood and lymph nodes. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are non-regulatory T cells. A conventional T cell commonly produces IL-2 and other interleukin factors. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are conventional T cells. In some embodiments, an isolated cell population comprising regulatory T cells comprises an undetectable amount of conventional T cells. In some embodiments, an isolated cell population comprising regulatory T cells comprises an undetectable amount of conventional CD4⁺ T cells. In some embodiments, an isolated cell population comprising regulatory T cells comprises an undetectable amount of B cells. In some embodiments, an isolated cell population comprising regulatory T cells comprises an undetectable amount of NK T cells. In some embodiments, an isolated cell population comprising regulatory T cells comprises an undetectable amount of CD14⁺ cells.

In some embodiments, an isolated cell population comprising regulatory T cells comprises an undetectable number of eosinophils. In some embodiments, an isolated cell population comprising regulatory T cells comprises an undetectable number of neutrophils. In some embodiments, an isolated population of cells comprising regulatory T cells comprises an undetectable amount of CD8⁺ T cells.

### Method of Culturing Regulatory T Cells

Some aspects of the present disclosure relate to methods of culturing an isolated cell population in cell media. The isolated cell populations comprising regulatory T cells may be cultured in such a way to increase the level of expansion of regulatory T cells following a period of time. In some embodiments, a method of culturing an isolated cell population comprising regulatory T cells involves culturing the cell population in the presence of one or more pro-inflammatory cytokines. For example, described herein is a method of culturing an isolated cell population comprising regulatory T cells in the presence of any one of pro-inflammatory cytokines selected from the group consisting of Interleukin-6 (IL-6), Interleukin-12 (IL-12), Tumor necrosis factor (TNF-α), Interleukin-17 (IL-17A), Interleukin-21 (IFN-β), Interferon gamma (IFN-γ), Transforming growth factor beta 1 (TGF-β1), and Interleukin-1b (IL-1β).

In some embodiments, the isolated cell population is cultured in the presence of one or more pro-inflammatory cytokines under activating conditions. In some embodiments, the isolated cell population is cultured in the presence of one or more pro-inflammatory cytokines and anti-CD3 and anti-CD28 antibodies. In such embodiments, the anti-CD3 and anti-CD28 antibodies function to activate the T cells of the isolated cell population. In some embodiments, the anti-CD3 and anti-CD8 antibodies are attached to a magnetic bead, such as CD3/CD28 Dynabeads™. In some embodiments, when the regulatory T cells express an exogenous TCR, the isolated cell population is cultured in the presence of one or more pro-inflammatory cytokines and a TCR ligand, e.g., a peptide MHC complex. In some embodiments, the peptide MHC complex is presented by an antigen presenting cell. In some embodiments, the peptide-MHC complex is presented by a multimer reagent, e.g., a tetramer or dextramer. In some embodiments, the isolated cell population is cultured in the presence of one or more pro-inflammatory cytokines, phorbol 12-myristate 13-acetate (PMA) and ionomycin to activate intracellular signaling pathways. In such embodiments, PMA and ionomycin function to activate the T cells of the isolated cell population through an activation of protein kinase C (PKC) and nuclear factor of activated T-cell (NFAT) signaling pathways. In some embodiments, the isolated cell population is activated after exposing the isolated cell population to pro-inflammatory conditions (e.g., culturing in the presence of one or more pro-inflammatory cytokines for a period of time). Such an activation may be helpful if the T cells of the isolated cell population begin to re-express CD3 following the initial activation before the exposure to pro-inflammatory conditions. In some embodiments, a step of activating the isolated cell population after exposure to pro-inflammatory conditions is performed prior to (e.g., immediately prior to) characterization of the isolated cell population (e.g., characterization of one or more markers of stability of regulatory T cells). In some embodiments, the isolated cell population is activated after exposing the isolated cell population to pro-inflammatory conditions using a combination of PMA and ionomycin.

In some embodiments, a method of culturing an isolated cell population comprising regulatory T cells involves culturing the cell population in the presence of IL-17A. In some embodiments, a method of culturing an isolated cell population comprising regulatory T cells involves culturing the cell population in the presence of IL-2 and IL-17A. In some embodiments, culturing an isolated cell population comprising regulatory T cells in the presence of IL-17A (e.g., IL-2 and IL-17A) results in an expanded cell population (e.g., an expanded cell population having higher abundance of regulatory T cells relative to a control, e.g., an isolated cell population cultured in the absence of IL-17A).

In some embodiments, a method of culturing an isolated cell population comprising regulatory T cells involves culturing the cell population in the presence of IFN-β. In some embodiments, a method of culturing an isolated cell population comprising regulatory T cells involves culturing the cell population in the presence of IL-2 and IFN-β. In some embodiments, culturing an isolated cell population comprising regulatory T cells in the presence of IFN-β (e.g., IL-2 and IFN-β) results in an expanded cell population (e.g., an expanded cell population having higher abundance of regulatory T cells relative to a control, e.g., an isolated cell population cultured in the absence of IFN-β).

In some embodiments, a method of culturing an isolated cell population comprising regulatory T cells involves culturing the cell population in the presence of IFN-γ. In some embodiments, a method of culturing an isolated cell population comprising regulatory T cells involves culturing the cell population in the presence of IL-2 and IFN-γ. In some embodiments, culturing an isolated cell population comprising regulatory T cells in the presence of IFN-γ (e.g., IL-2 and IFN-γ) results in an expanded cell population (e.g., an expanded cell population having higher abundance of regulatory T cells relative to a control, e.g., an isolated cell population cultured in the absence of IFN-γ).

In some embodiments, a method of culturing an isolated cell population comprising regulatory T cells involves culturing the cell population in the presence of IL-1β. In some embodiments, a method of culturing an isolated cell population comprising regulatory T cells involves culturing the cell population in the presence of IL-2 and IL-1β. In some embodiments, culturing an isolated cell population comprising regulatory T cells in the presence of IL-1β (e.g., IL-2 and IL-1β) results in an expanded cell population (e.g., an expanded cell population having higher abundance of regulatory T cells relative to a control, e.g., an isolated cell population cultured in the absence of IL-1β).

In some embodiments, a method of culturing an isolated cell population comprising regulatory T cells involves culturing the cell population in the presence of TNF-α. In some embodiments, a method of culturing an isolated cell population comprising regulatory T cells involves culturing the cell population in the presence of IL-2 and TNF-α. In some embodiments, culturing an isolated cell population comprising regulatory T cells in the presence of TNF-α (e.g., IL-2 and TNF-α) results in an expanded cell population (e.g., an expanded cell population having higher abundance of regulatory T cells relative to a control, e.g., an isolated cell population cultured in the absence of TNF-α).

In some embodiments, a method of culturing an isolated cell population comprising regulatory T cells involves culturing the cell population in the presence of IL-2 and at least three additional pro-inflammatory cytokines. In some embodiments, culturing an isolated cell population comprising regulatory T cells in the presence of IL-2 and at least three additional pro-inflammatory cytokines results in an expanded cell population (e.g., an expanded cell population having higher abundance of regulatory T cells relative to a control, e.g., an isolated cell population cultured in the absence of IL-2 and at least three additional pro-inflammatory cytokines).

In some embodiments, a method of culturing an isolated cell population comprising the regulatory T cell in the presence of IL-2, IL-6, and at least two (e.g., 2, 3, 4, 5, 6, or 7) additional pro-inflammatory cytokines selected from: IL-12, TNF-α, IL-17A, IFN-β, IFN-γ, TGF-β1, and IL-1β. In some embodiments, a method of culturing an isolated cell population comprising the regulatory T cell in the presence of IL-2, IL-12, and at least two (e.g., 2, 3, 4, 5, 6, or 7) additional pro-inflammatory cytokines selected from: IL-6, TNF-α, IL-17A, IFN-β, IFN-γ, TGF-β1, and IL-1β. In some embodiments, a method of culturing an isolated cell population comprising the regulatory T cell in the presence of IL-2, TNF-α, and at least two (e.g., 2, 3, 4, 5, 6, or 7) additional pro-inflammatory cytokines selected from: IL-6, IL-12, IL-17A, IFN-β, IFN-γ, TGF-β1, and IL-1β. In some embodiments, a method of culturing an isolated cell population comprising the regulatory T cell in the presence of IL-2, IL-17A, and at least two (e.g., 2, 3, 4, 5, 6, or 7) additional pro-inflammatory cytokines selected from: IL-6, IL-12, TNF-α, IFN-β, IFN-γ, TGF-β1, and IL-1β. In some embodiments, a method of culturing an isolated cell population comprising the regulatory T cell in the presence of IL-2, IFN-β, and at least two (e.g., 2, 3, 4, 5, 6, or 7) additional pro-inflammatory cytokines selected from: IL-6, IL-12, TNF-α, IL-17A, IFN-γ, TGF-β1, and IL-1β. In some embodiments, a method of culturing an isolated cell population comprising the regulatory T cell in the presence of IL-2, IFN-γ, and at least two (e.g., 2, 3, 4, 5, 6, or 7) additional pro-inflammatory cytokines selected from: IL-6, IL-12, TNF-α, IL-17A, IFN-β, TGF-β1, and IL-1β. In some embodiments, a method of culturing an isolated cell population comprising the regulatory T cell in the presence of IL-2, TGF-β1, and at least two (e.g., 2, 3, 4, 5, 6, or 7) additional pro-inflammatory cytokines selected from: IL-6, IL-12, TNF-α, IL-17A, IFN-β, IFN-γ, and IL-1β. In some embodiments, a method of culturing an isolated cell population comprising the regulatory T cell in the presence of IL-2, IL-1β, and at least two (e.g., 2, 3, 4, 5, 6, or 7) additional pro-inflammatory cytokines selected from: IL-6, IL-12, TNF-α, IL-17A, IFN-β, IFN-γ, and TGF-β1.

In some embodiments, a method of culturing an isolated cell population comprising the regulatory T cell in the presence of IL-2, IL-1β, IL-6, IL-12, TNF-α, IL-17A, IFN-β, IFN-γ, and TGF-β1.

In some embodiments, a method of culturing an isolated cell population comprising the regulatory T cell in the presence of IL-2, IL-1β, IL-6, IL-12, IL-17A, IFN-γ, TNF-α, IFN-β, and TGF-β1.

In some embodiments, a method of culturing an isolated cell population comprising the regulatory T cell in the presence of IL-2, IL-1β, IL-6, IL-12, IL-17A, IFN-γ, and TNF-α.

The concentration of any one of the pro-inflammatory cytokines (e.g., IL-2, IL-1β, IL-6, IL-12, TNF-α, IL-17A, IFN-β, IFN-γ, and TGF-β1) may be in the range of 0.01-10000 pg/mL, 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL. In some embodiments, the concentration of any one of the pro-inflammatory cytokines is about 500, about 1000, about 1500, or about 2000 pg/ml.

In some embodiments, the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL. In some embodiments, the concentration of IL-2 is about 500, about 1000, about 1500, or about 2000 U/ml.

In some embodiments, the concentration of IL-1β, IL-6, IL-12, TNF-α, IL-17A, IFN-β, IFN-γ, or TGF-1 is in the range of 1-10000 pg/mL, 1-1000, pg/mL, or 100-1000 pg/mL. In some embodiments, the concentration of IL-1β, IL-6, IL-12, TNF-α, IL-17A, IFN-β, IFN-γ, or TGF-β1 is about 1000 pg/mL. In some embodiments, the concentration of IL-2 is about 100-2000 U/mL and the concentration of IL-1β is about 100-2000 pg/mL. In some embodiments, the concentration of IL-2 is about 500-1500 U/mL and the concentration of IL-1β is about 500-1500 pg/mL. In some embodiments, the concentration of IL-2 is about 1000 U/mL and the concentration of IL-1B is about 1000 pg/mL. In some embodiments, the concentration of IL-2 is about 100-2000 U/mL and the concentration of IL-6 is about 100-2000 pg/mL. In some embodiments, the concentration of IL-2 is about 500-1500 U/mL and the concentration of IL-6 is about 500-1500 pg/mL. In some embodiments, the concentration of IL-2 is about 1000 U/mL and the concentration of IL-6 is about 1000 pg/mL. In some embodiments, the concentration of IL-2 is about 100-2000 U/mL and the concentration of IL-12 is about 100-2000 pg/mL. In some embodiments, the concentration of IL-2 is about 500-1500 U/mL and the concentration of IL-12 is about 500-1500 pg/mL. In some embodiments, the concentration of IL-2 is about 1000 U/mL and the concentration of IL-12 is about 1000 pg/mL. In some embodiments, the concentration of IL-2 is about 100-2000 U/mL and the concentration of TNF-α is about 100-2000 pg/mL. In some embodiments, the concentration of IL-2 is about 500-1500 U/mL and the concentration TNF-α is about 500-1500 pg/mL. In some embodiments, the concentration of IL-2 is about 1000 U/mL and the concentration of TNF-α is about 1000 pg/mL. In some embodiments, the concentration of IL-2 is about 100-2000 U/mL and the concentration of IL-17A is about 100-2000 pg/mL. In some embodiments, the concentration of IL-2 is about 500-1500 U/mL and the concentration of IL-17A is about 500-1500 pg/mL. In some embodiments, the concentration of IL-2 is about 1000 U/mL and the concentration of IL-17A is about 1000 pg/mL. In some embodiments, the concentration of IL-2 is about 100-2000 U/mL and the concentration of IFN-β is about 100-2000 pg/mL. In some embodiments, the concentration of IL-2 is about 500-1500 U/mL and the concentration of IFN-β is about 500-1500 pg/mL. In some embodiments, the concentration of IL-2 is about 1000 U/mL and the concentration of IFN-β is about 1000 pg/mL. In some embodiments, the concentration of IL-2 is about 100-2000 U/mL and the concentration of IFN-γ is about 100-2000 pg/mL. In some embodiments, the concentration of IL-2 is about 500-1500 U/mL and the concentration of IFN-γ is about 500-1500 pg/mL. In some embodiments, the concentration of IL-2 is about 1000 U/mL and the concentration of IFN-γ is about 1000 pg/mL. In some embodiments, the concentration of IL-2 is about 100-2000 U/mL and the concentration of TGF-β1 is about 100-2000 pg/mL. In some embodiments, the concentration of IL-2 is about 500-1500 U/mL and the concentration of TGF-β1 is about 500-1500 pg/mL. In some embodiments, the concentration of IL-2 is about 1000 U/mL and the concentration of TGF-β1 is about 1000 pg/mL.

Without being bound by any theory, any one of the pro-inflammatory cytokines may be capable of inducing a signaling cascade within the regulatory T cells of the isolated cell population. This signaling cascade may be responsible, at least in part, with causing an increased expansion of regulatory T cells in the presence of the one or more pro-inflammatory cytokines.

As described herein, the level of expansion of an isolated cell population may be determined relative to a control. In some embodiments, the level of expansion of an isolated cell population may be determined relative to a control, wherein the control is a control isolated cell population that is not being cultured in the presence of additional pro-inflammatory cytokines. In some embodiments, the level of expansion of an isolated cell population may be determined relative to a control, wherein the control is the cell population prior to incubation with the one or more pro-inflammatory cytokines.

Culturing the isolated cell population may involve culturing the isolated cell population in a cell media. In some embodiments, the cell media may be replaced and/or supplemented with new or fresh cell media at regular intervals. In some embodiments, the cell media may be replaced and/or supplemented with new or fresh cell media every day, every second day, every third day, every fourth day, every fifth day, every six days, or once per week. In some embodiments, the cell media may be replaced and/or supplemented with new or fresh cell media every 1-5 days, 1-4 days, 1-3 days, 1-2 days, 2-5 days, 2-4 days, 2-3 days, 3-5 days, or 3-4 days.

An isolated cell population comprising regulatory T cells may be cultured in the presence of pro-inflammatory cytokines for at least 3, at least 4, at least 5 at least 6, at least 7, at least 8, at least 9, at least 10, at least 11, at least 12, at least 15, at least 20, at least 23, at least 25, or at least 30 days. In some embodiments, an isolated cell population comprising regulatory T cells may be cultured in the presence of pro-inflammatory cytokines for 1-30 days, 2-30 days, 2-20 days, 2-10 days, 5-20 days, 5-10 days, 3-25 days, 5-25 days, 5-20 days, 10-20 days, 10-25 days, 5-10 days, 10-30 days, 10-25 days, 10-20 days, 15-30 days, 20-30 days, 10-60 days, 10-50 days, 10-40 days, or 20-40 days. In some embodiments, an isolated cell population comprising regulatory T cells may be cultured in the presence of pro-inflammatory cytokines for is 1, 2, 3, 4, 5, 6, 7, 8, 9, 10, 11, 12, 13, 14, 20, 23, 25, or 30 days. In some embodiments, a period of time is 1-6 weeks, 1-5 weeks, 1-4 weeks, 1-3 weeks, 1-2 weeks, or 2-4 weeks.

A cell media for use herein may comprise a buffering agent, salts, a protein source, lipids, and/or essential cell nutrients. In some embodiments, the cell media comprises phosphate-buffered saline (PBS), bovine serum albumin, human serum albumin, and/or EDTA.

An isolated cell population may be cultured in the presence of antigen-presenting cells or agents that mimic antigen-presenting cells. An agent that mimics antigen-presenting cells may include three-dimensional beads (e.g., spherical beads). In some embodiments, the three-dimensional beads are attached to activation signals such as CD3, CD28, and/or CD137. In some embodiments, the agents that mimic antigen-presenting cells are human T-activator CD3/CD28 Dynabeads™. In some embodiments, the agents that mimic antigen-presenting cells comprise anti-CD3 and/or anti-CD28 antibodies.

In some embodiments, cell media comprises RPMI1640, human or bovine serum, L-glutamine, penicillin-streptomycin, β-mercaptoethanol (50 μM), sodium pyruvate, nonessential amino acids, and/or a buffer (e.g., PBS or HEPES). In some embodiments, cell media comprises a commercial product.

In some embodiments, an isolated cell population comprising regulatory T cells may be a modified version of the culture conditions may be found in Chakraborty R, et al. Robust and cost-effective expansion of human regulatory T cells highly functional in a xenograft model of graft-versus-host disease. Haematologica. 2013 April;98(4):533-7. doi: 10.3324/haematol.2012.076430. Epub 2012 Dec. 14. PMID: 23242592; PMCID: PMC3659983 or Siemasko, K. In Vitro Expanded CD4+CD25+Foxp3+Regulatory T Cells Maintain a Normal Phenotype and Suppress Immune-Mediated Ocular Surface Inflammation. Invest. Ophthalmol. Vis. Sci. 2008; 49(12):5434-5440. doi: doi.org/10.1167/iovs.08-2075.

### ADDITIONAL EMBODIMENTS

Additional embodiments of the disclosure are set forth in the following numbered paragraphs:

1 A method of evaluating the stability profile of an isolated cell population comprising regulatory T cells, the method comprising:
(i) culturing the isolated cell population in the presence of two or more pro-inflammatory cytokines for a period of time;
(ii) characterizing one or more markers of stability of the isolated cell population following the period of time; and
(iii) evaluating the stability profile of the isolated cell population based on the characterizing of the one or more markers of stability.

2. A method comprising:
(i) culturing an isolated cell population comprising regulatory T cells in the presence of two or more pro-inflammatory cytokines for a period of time; and
(ii) characterizing one or more markers of stability of the isolated cell population following the period of time.

3. The method of paragraph 1 or 2, wherein the two or more pro-inflammatory cytokines comprises at least two, at least three, at least four, at least five, at least six, at least seven, at least eight, or at least nine cytokines selected from the group consisting of Interleukin-2 (IL-2), Interleukin-6 (IL-6), Interleukin-12 (IL-12), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

4. The method of any one of the preceding paragraphs, wherein the two or more pro-inflammatory cytokines comprises IL-2 and at least one, at least two, at least three, at least four, at least five, at least six, at least seven, or at least eight cytokines selected from the group consisting of IL-6, IL-12, TNF-α, IL-17A, IFN-β, IFN-γ, TGF-β1, and IL-1β.

5. The method of any one of the preceding paragraphs, wherein the two or more pro-inflammatory cytokines comprises:
(a) IL-2 and IL-6;
(b) IL-2 and IL-12;
(c) IL-2 and TNF-α;
(d) IL-2 and IL-17A;
(e) IL-2 and IFN-β;
(f) IL-2 and IFN-γ;
(g) IL-2 and TGF-β1; or
(h) IL-2 and IL-1β.

6. The method of any one of the preceding paragraphs, wherein the two or more pro-inflammatory cytokines comprises:
(a) IL-2, IL-6, and at least one additional pro-inflammatory cytokine selected from: IL-12, TNF-α, IL-17A, IFN-β, IFN-γ, TGF-β1, and IL-1β;
(b) IL-2, IL-12, and at least one additional pro-inflammatory cytokine selected from: IL-6, TNF-α, IL-17A, IFN-β, IFN-γ, TGF-β1, and IL-1β;
(c) IL-2, TNF-α, and at least one additional pro-inflammatory cytokine selected from: IL-6, IL-12, IL-17A, IFN-β, IFN-γ, TGF-β1, and IL-1β;
(d) IL-2, IL-17A, and at least one additional pro-inflammatory cytokine selected from: IL-6, IL-12, TNF-α, IFN-β, IFN-γ, TGF-β1, and IL-1β;
(e) IL-2, IFN-β, and at least one additional pro-inflammatory cytokine selected from: IL-6, IL-12, TNF-α, IL-17A, IFN-γ, TGF-β1, and IL-1β;
(f) IL-2, IFN-γ, and at least one additional pro-inflammatory cytokine selected from: IL-6, IL-12, TNF-α, IL-17A, IFN-β, TGF-β1, and IL-1β;
(g) IL-2, TGF-β1, and at least one additional pro-inflammatory cytokine selected from: IL-6, IL-12, TNF-α, IL-17A, IFN-β, IFN-γ, and IL-1β; or
(h) IL-2, IL-1β, and at least one additional pro-inflammatory cytokine selected from: IL-6, IL-12, TNF-α, IL-17A, IFN-β, IFN-γ, and TGF-β1.

7. The method of any one of the preceding paragraphs, wherein the two or more pro-inflammatory cytokines comprises:
(a) IL-2, IL-6, and at least two additional pro-inflammatory cytokines selected from: IL-12, TNF-α, IL-17A, IFN-β, IFN-γ, TGF-β1, and IL-1β;
(b) IL-2, IL-12, and at least two additional pro-inflammatory cytokines selected from: IL-6, TNF-α, IL-17A, IFN-β, IFN-γ, TGF-β1, and IL-1β;
(c) IL-2, TNF-α, and at least two additional pro-inflammatory cytokines selected from: IL-6, IL-12, IL-17A, IFN-β, IFN-γ, TGF-β1, and IL-1β;
(d) IL-2, IL-17A, and at least two additional pro-inflammatory cytokines selected from: IL-6, IL-12, TNF-α, IFN-β, IFN-γ, TGF-β1, and IL-1β;
(e) IL-2, IFN-β, and at least two additional pro-inflammatory cytokines selected from: IL-6, IL-12, TNF-α, IL-17A, IFN-γ, TGF-β1, and IL-1β;
(f) IL-2, IFN-γ, and at least two additional pro-inflammatory cytokines selected from: IL-6, IL-12, TNF-α, IL-17A, IFN-β, TGF-β1, and IL-1β;
(g) IL-2, TGF-β1, and at least two additional pro-inflammatory cytokines selected from: IL-6, IL-12, TNF-α, IL-17A, IFN-β, IFN-γ, and IL-1β; or
(h) IL-2, IL-1β, and at least two additional pro-inflammatory cytokines selected from: IL-6, IL-12, TNF-α, IL-17A, IFN-β, IFN-γ, and TGF-β1.

8. The method of any one of the preceding paragraphs, wherein the two or more pro-inflammatory cytokines comprises:
(a) IL-2, IL-6, and at least three additional pro-inflammatory cytokines selected from: IL-12, TNF-α, IL-17A, IFN-β, IFN-γ, TGF-β1, and IL-1β;
(b) IL-2, IL-12, and at least three additional pro-inflammatory cytokines selected from: IL-6, TNF-α, IL-17A, IFN-β, IFN-γ, TGF-β1, and IL-1β;
(c) IL-2, TNF-α, and at least three additional pro-inflammatory cytokines selected from: IL-6, IL-12, IL-17A, IFN-β, IFN-γ, TGF-β1, and IL-1β;
(d) IL-2, IL-17A, and at least three additional pro-inflammatory cytokines selected from: IL-6, IL-12, TNF-α, IFN-β, IFN-γ, TGF-β1, and IL-1β;
(e) IL-2, IFN-β, and at least three additional pro-inflammatory cytokines selected from: IL-6, IL-12, TNF-α, IL-17A, IFN-γ, TGF-β1, and IL-1β;
(f) IL-2, IFN-γ, and at least three additional pro-inflammatory cytokines selected from: IL-6, IL-12, TNF-α, IL-17A, IFN-β, TGF-1, and IL-1β;
(g) IL-2, TGF-β1, and at least three additional pro-inflammatory cytokines selected from: IL-6, IL-12, TNF-α, IL-17A, IFN-β, IFN-γ, and IL-1β; or
(h) IL-2, IL-1β, and at least three additional pro-inflammatory cytokines selected from: IL-6, IL-12, TNF-α, IL-17A, IFN-β, IFN-γ, and TGF-1.

9 The method of any one of the preceding paragraphs, wherein the two or more pro-inflammatory cytokines comprises IL-2, IL-6, IL-12, TNF-α, IL-17A, IFN-β, IFN-γ, TGF-β1, and IL-1β.

10. The method of any one of paragraph 1-4, wherein the two or more pro-inflammatory cytokines comprise IL-2, IL-1β, IL-6, IL-12, IL-17A, IFN-γ, TNF-α, IFN-β, and TGF-β1, optionally wherein the concentration of each of IL-1β, IL-6, IL-12, IL-17A, IFN-γ, TNF-α, IFN-β, and TGF-β1 is about 1000 pg/mL, optionally wherein the concentration of IL-2 is 1000 U/mL.

11. The method of any one of paragraph 1-4, wherein the two or more pro-inflammatory cytokines comprise IL-2, IL-1β, IL-6, IL-12, IL-17A, IFN-γ, and TNF-α, optionally wherein the concentration of each of IL-1β, IL-6, IL-12, IL-17A, IFN-γ, and TNF-αis about 1000 pg/mL, optionally wherein the concentration of IL-2 is 1000 U/mL.

12. The method of any one of the preceding paragraphs, wherein the two or more pro-inflammatory cytokines further comprise Interleukin-22 (IL-22), Granulocyte-macrophage colony-stimulating factor (GM-CSF) and/or Interleukin-23 (IL-23).

13. The method of any one of the preceding paragraphs, wherein the concentration of any one of the pro-inflammatory cytokines is in the range of 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

14. The method of any one of paragraphs 3-13, wherein the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL.

15. The method of any one of the preceding paragraphs, wherein the period of time is at least 3, at least 6, at least 9, at least 12, at least 15, at least 20, or at least 25 days.

16. The method of any one of the preceding paragraphs, wherein the period of time is 3 days, 4 days, 5 days, 6 days, 7 days, 8 days, or 9 days.

17. The method of any one of paragraphs 1-14, wherein the period of time is 2-30 days, 3-25 days, 5-25 days, 5-20 days, 5-10 days, 10-30 days, 10-25 days, 10-20 days, 15-30 days, or 20-30 days.

18. The method of any one of the preceding paragraphs further comprising characterizing the one or more markers of stability of the isolated cell population prior to (i).

19. The method of any one of the preceding paragraphs further comprising activating the isolated cell population between steps (i) and (ii), optionally wherein activating the isolated cell population is performed by incubating the cell population with phorbol 12-myristate 13-acetate (PMA) and ionomycin.

20. The method of any one of the preceding paragraphs further comprising characterizing the one or more markers of stability of the isolated cell population at regular intervals throughout the period of time.

21. The method of paragraph 20, wherein the method comprises characterizing the one or more markers of stability of the isolated cell population every day, every second day, every third day, or every fourth day throughout the period of time.

22. The method of any one of the preceding paragraphs, wherein characterizing one or more markers of stability comprises determining expression and/or activity of the one or more markers of stability.

23. The method of paragraph 22, wherein determining expression and/or activity of the one or more markers of stability is performed using biochemical techniques.

24. The method of paragraph 22, wherein determining expression and/or activity of the one or more markers of stability is performed using fluorescence activated cell sorting (FACS).

25. The method of any one of the preceding paragraphs, wherein the one or more markers of stability comprise the biomarkers CD4, CD25, and/or FOXP3.

26. The method of any one of the preceding paragraphs, wherein the one or more markers of stability comprise the methylation of a regulatory T cell-specific demethylation region (TSDR) at an endogenous FOXP3 locus and/or FOXP3 expression.

27. The method of any one of the preceding paragraphs further comprising characterizing the relative abundance of IFN-γ⁺ cells, IL-17⁺ cells, and/or TNF-α⁺ cells within the cell population after (i).

28. The method of any one of the preceding paragraphs, wherein at least 80% of the cells of the isolated cell population are CD25^{+/high}CD4⁺CD127^{-/lo} regulatory T cells.

29. The method of any one of the preceding paragraphs, wherein, prior to the culturing of step (i), at least 70% of the cells of the isolated cell population are stable regulatory T cells comprising a hypomethylated TSDR at an endogenous FOXP3 locus and/or at least 80% of the cells of the isolated cell population are FOXP3+.

30. The method of any one of the preceding paragraphs, wherein the isolated cell population has undergone one or more cryopreservation freeze-thaw cycles.

31. The method of any one of the preceding paragraphs, wherein culturing the isolated cell population comprises culturing the isolated cell population in a cell media.

32. The method of paragraph 31, wherein culturing the isolated cell population comprises replacing the cell media with new cell media at regular intervals.

33. The method of paragraph 31 or 32, wherein the cell media comprises a buffer.

34. The method of any one of paragraphs 31-33, wherein the cell media comprises phosphate-buffered saline (PBS), bovine serum albumin, human serum albumin, and/or EDTA.

35. The method of any one of the preceding paragraphs, wherein the isolated cell population is cultured in the presence of one or more agents that activate T cells within the isolated cell population, optionally wherein the one or more agents are antigen-presenting cells or agents that mimic antigen-presenting cells.

36. The method of paragraph 35, wherein the agents that mimic antigen-presenting cells are three-dimensional beads, optionally wherein the three-dimensional beads are attached to activation signals.

37. The method of paragraph 35 or 36, wherein the agents that mimic antigen-presenting cells are three-dimensional beads that are attached to anti-CD3, anti-CD28, and/or anti-CD137 molecules, optionally wherein the agents are polystyrene beads coated with a mixture of anti-CD3 and/or anti-CD28 antibodies.

38. The method of paragraph 35, wherein the one or more agents that activate T cells are phorbol 12-myristate 13-acetate (PMA) and ionomycin.

39. The method of any one of paragraphs 31-37, wherein culturing the isolated cell population comprises replacing the cell media with new cell media every 2-5 days, optionally every 3-4 days.

40. The method of any one of the preceding paragraphs, wherein the isolated cell population is a stable cell population if at least 80% of the regulatory T cells comprise a hypomethylated TSDR and an endogenous FOXP3 locus and/or at least 80% of the cells of the isolated cell population are FOXP3+.

41. A method of expanding a cell population, the method comprising culturing an isolated cell population comprising regulatory T cells in the presence of IL-2 and at least three additional pro-inflammatory cytokines.

42. The method of paragraph 41, wherein the at least three additional pro-inflammatory cytokines are selected from the group consisting of Interleukin-6 (IL-6), Interleukin-12 (IL-12), Tumor necrosis factor (TNF-α), Interleukin-17 (IL-17A), Interleukin-21 (IFN-β), Interferon gamma (IFN-γ), Transforming growth factor beta 1 (TGF-β1), and Interleukin-1b (IL-1β).

43. The method of paragraph 41 or 42, wherein the at least three additional pro-inflammatory cytokine comprises at least four, at least five, at least six, at least seven, or at least eight additional pro-inflammatory cytokines selected from the group consisting of Interleukin-6 (IL-6), Interleukin-12 (IL-12), Tumor necrosis factor (TNF-α), Interleukin-17 (IL-17A), Interferon beta (IFN-β), Interferon gamma (IFN-γ), Transforming growth factor beta 1 (TGF-β1), and Interleukin-1b (IL-1β).

44. The method of any one of paragraphs 41-43, wherein the at least three additional pro-inflammatory cytokines comprise IL-6, IL-12, TNF-α, IL-17A, IFN-β, IFN-γ, TGF-β1, and IL-1β.

45. A method of expanding a cell population, the method comprising culturing an isolated cell population comprising regulatory T cells in the presence of Interleukin-17 (IL-17A).

46. A method of expanding a cell population, the method comprising culturing an isolated cell population comprising regulatory T cells in the presence of Interferon beta (IFN-β).

47. A method of expanding a cell population, the method comprising culturing an isolated cell population comprising regulatory T cells in the presence of Interferon gamma (IFN-γ).

48. A method of expanding a cell population, the method comprising culturing an isolated cell population comprising regulatory T cells in the presence of Interleukin-1b (IL-1β).

49. A method of expanding a cell population, the method comprising culturing an isolated cell population comprising regulatory T cells in the presence of Tumor necrosis factor alpha (TNF-α) and IL-2.

50. The method of any one of paragraphs 45-48 further comprising culturing the isolated cell population in the presence of one or more additional pro-inflammatory cytokines.

51. The method of paragraph 50, wherein the one or more additional pro-inflammatory cytokines is Interleukin-2 (IL-2), Interleukin-6 (IL-6), Interleukin-12 (IL-12), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

52. The method of any one of paragraphs 41-51, wherein the concentration of any one of the pro-inflammatory cytokines is in the range of 0.01-10000 pg/mL, 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

53. The method of any one of paragraphs 41-52, wherein the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL, optionally wherein the concentration of IL-2 is 1000 U/mL.

54. The method of any one of paragraphs 41-53, wherein the concentration of each of the at least three additional pro-inflammatory cytokines is in the range of 100-1000 pg/mL.

55. The method of any one of paragraphs 41-54, wherein the pro-inflammatory cytokines induce a signaling cascade within the regulatory T cells of the isolated cell population.

56. The method of any one of paragraphs 41-55, wherein the level of expansion of the isolated cell population is determined relative to a control, optionally wherein the control is a control isolated cell population that is not being cultured in the presence of additional pro-inflammatory cytokines.

57. The method of paragraph 56, wherein the level of expansion of the isolated cell population is increased relative to the control.

58. The method of any one of paragraphs 41-57, wherein at least 80% of the cells of the isolated cell population are CD25^{+/high}CD4⁺CD127^{-/lo} regulatory T cells.

59. The method of any one of paragraphs 41-58, wherein the isolated cell population is a stable cell population if at least 80% of the regulatory T cells comprise a hypomethylated TSDR and an endogenous FOXP3 locus.

60. The method of any one of paragraphs 41-59, wherein the isolated cell population has undergone one or more cryopreservation freeze-thaw cycles prior to culturing.

61. The method of any one of paragraphs 41-60, wherein culturing the isolated cell population comprises culturing the isolated cell population in a cell media.

62. The method of paragraph 61, wherein culturing the isolated cell population comprises replacing the cell media with new cell media at regular intervals.

63. The method of paragraph 61 or 62, wherein culturing the isolated cell population comprises replacing the cell media with new cell media every 2-5 days, optionally every 3-4 days.

64. The method of any one of paragraphs 41-63, wherein culturing the isolated cell population comprises culturing the isolated cell population for at least 3, at least 6, at least 9, at least 12, at least 15, at least 20, or at least 25 days.

65. The method of any one of paragraphs 41-63, wherein culturing the isolated cell population comprises culturing the isolated cell population for 2-30 days, 3-25 days, 5-25 days, 5-20 days, 5-10 days, 10-30 days, 10-25 days, 10-20 days, 15-30 days, or 20-30 days.

66. The method of any one of paragraphs 41-65, further comprising isolating the cell population.

67. The method of any one of paragraphs 61-66, wherein the cell media comprises a buffer.

68. The method of any one of paragraphs 61-67, wherein the cell media comprises phosphate-buffered saline (PBS), bovine serum albumin, human serum albumin, and/or EDTA.

69. The method of any one of paragraphs 41-68, wherein the isolated cell population is cultured in the presence of one or more agents that activate T cells within the isolated cell population, optionally wherein the one or more agents are antigen-presenting cells or agents that mimic antigen-presenting cells.

70. The method of paragraph 69, wherein the agents that mimic antigen-presenting cells are three-dimensional beads, optionally wherein the three-dimensional beads are attached to activation signals.

71. The method of paragraph 69 or 70, wherein the agents that mimic antigen-presenting cells are three-dimensional beads are attached to anti-CD3, anti-CD28, and/or anti-CD137, optionally wherein the agents are polystyrene beads coated with a mixture of anti-CD3 and/or anti-CD28 antibodies.

### EXAMPLES

### Example 1

Pro-inflammatory cytokines for use in this Example were selected based on their abundance in serum and CSF samples of multiple sclerosis patients and the mRNA expression levels of their corresponding receptors in human Tregs.

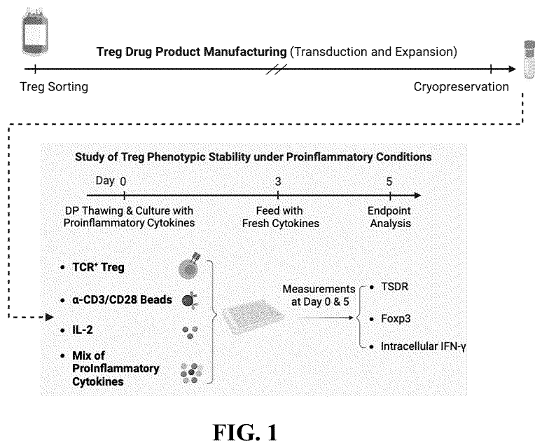

FIG. 1 provides a schematic of a regulatory T cell stability assay of the disclosure, described below.

For the experiments described in this Example, CD4⁺ CD25⁺ CD127^{low} regulatory T cells were first isolated from PBMCs from a healthy donor using immunomagnetic cell isolation kit (StemCell Technologies, Cat#18063). Flow cytometry was then used to sort for CD4⁺ CD25⁺ CD127^{low}CD45RA+ Tregs (FACS). Sorted regulatory T cells were expanded for 8 days in the presence of human T-activator CD3/CD28 Dynabeads™ (Life Technologies, Cat#11132D), cell culture medium, and human recombinant IL-2. Post expansion, the regulatory T cell population was characterized using flow cytometry and droplet digital PCR (ddPCR) for TSDR analysis to assess the purity and stability of the cells, respectively. The regulatory T cell population was then cryopreserved.

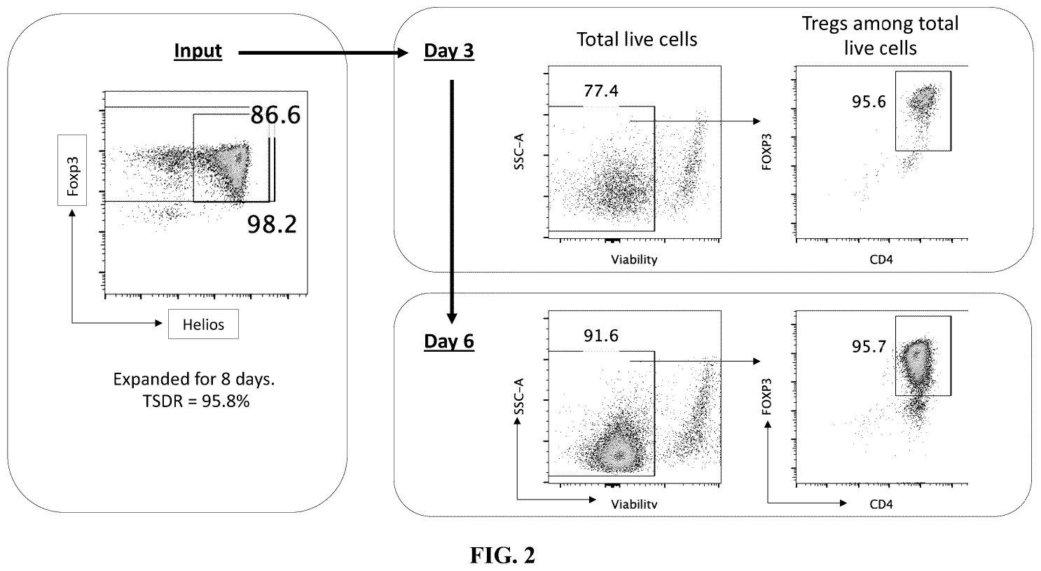

To assess the stability of the regulatory T cells under inflammatory conditions (using a stability assay of the disclosure), the regulatory T cells were thawed and plated into 96 round bottom tissue-culture-treated plates at 50,000 cells per well in 200 μL X-VIVO 15 serum-free hematopoietic cell medium (Lonza, Cat#04-418Q). Individual wells were supplemented with (a) human recombinant IL-2, (b) IL-2 and one of the following pro-inflammatory cytokines: IL-1β, IL-6, IL-12, IL-17A, IFN-β, IFN-γ, TGF-β1, or TNF-α, or (c) a mixture of IL-2, IL-1β, IL-6, IL-12, IL-17A, IFN-β, IFN-γ, TGF-β1, and TNF-α. In each experiment, the concentration of IL-2 remained the same (1000 U/mL), while the other pro-inflammatory cytokines varied (0.01, 0.1, 1, 10, 100, 1000, or 10000 pg/mL). The cells were split (1:2) every 3 to 4 days (Day 3, Day 6, Day 9, etc.) and activated with 25,000 T-activator CD3/CD28 Dynabeads™. Each time the cells were split, the media was replaced with fresh X-VIVO 15 media supplemented with fresh CD3/CD28 Dynabeads™ and cytokines. At Day 0, Day 3, Day 6, and Day 9, FACS-based immunophenotyping was used to characterize the cells (FIG. 2). At Day 0 (“Input”), cells were detected based on expression of FoxP3 and Helios expression. Cell expansion was also measured. At the endpoint, cells were collected for FACS analysis, and ddPCR was used to measure TSDR demethylation.

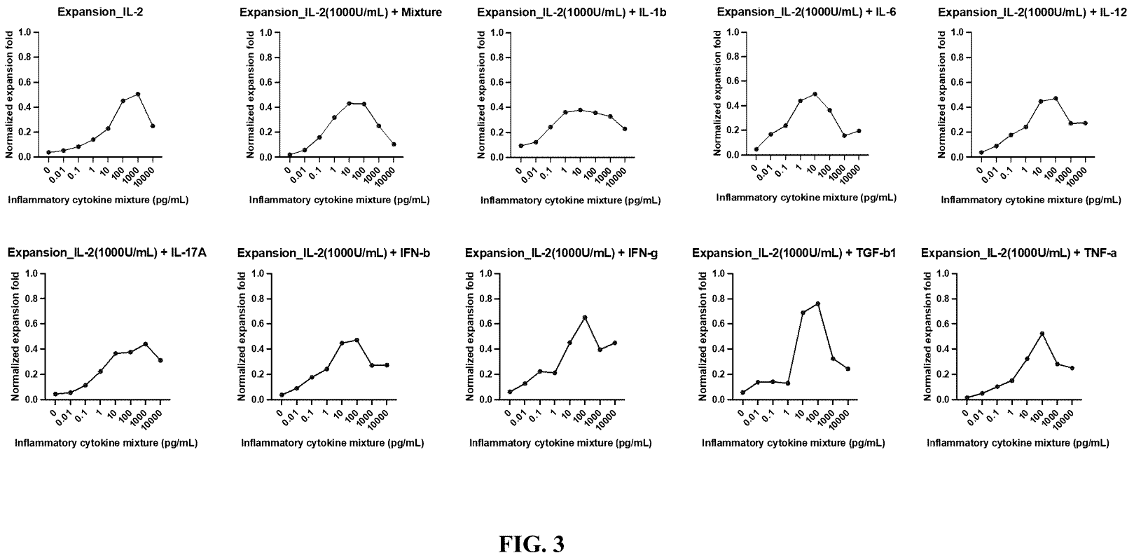

The data show that culturing regulatory T cells in the presence of IL-2 and at least one of the additional cytokines (IL-1β, IL-6, IL-12, IL-17A, IFN-β, IFN-γ, TGF-β1, TNF-α, or the complete mixture) resulted in an improved level of regulatory T cell expansion, relative to a control, particularly when the concentration of the second cytokine was 10-1000 pg/mL (FIG. 3).

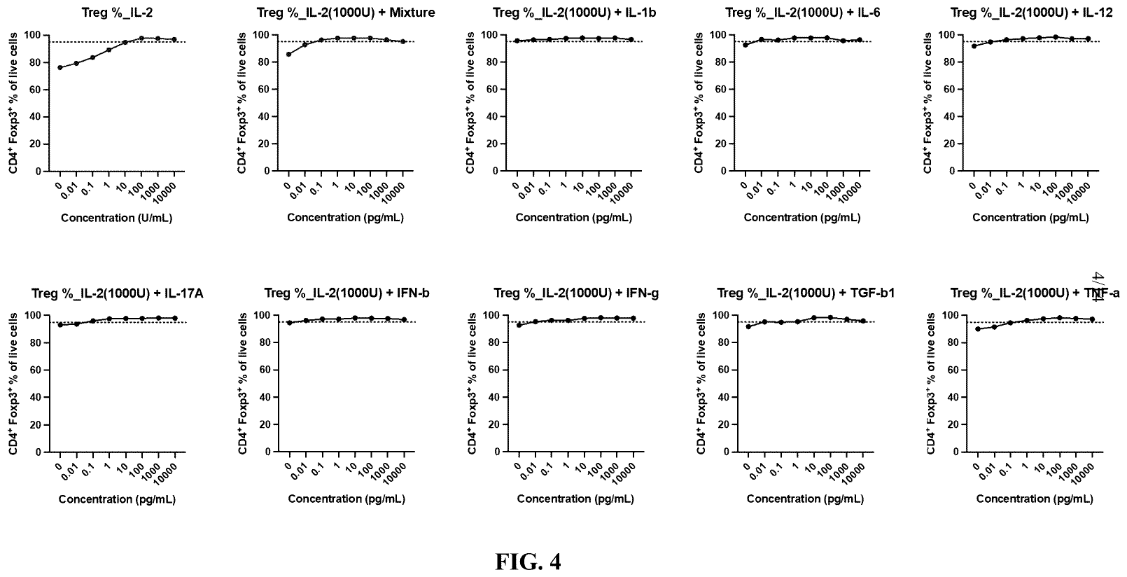

The stability profile of the regulatory T cells was also assessed by determining the percentage of live cells that are CD4+ cells expressing the Treg marker FOXP3 (viable CD4⁺ Foxp3⁺ cells). The data show that the regulatory T cells, in the presence of IL-2 and at least one additional cytokine from the mixture, at the concentrations shown, retain expression of FOXP3 at each of the cytokine concentrations tested, indicating that the cells stably retain a Treg phenotype in the presence of the cytokines (FIG. 4).

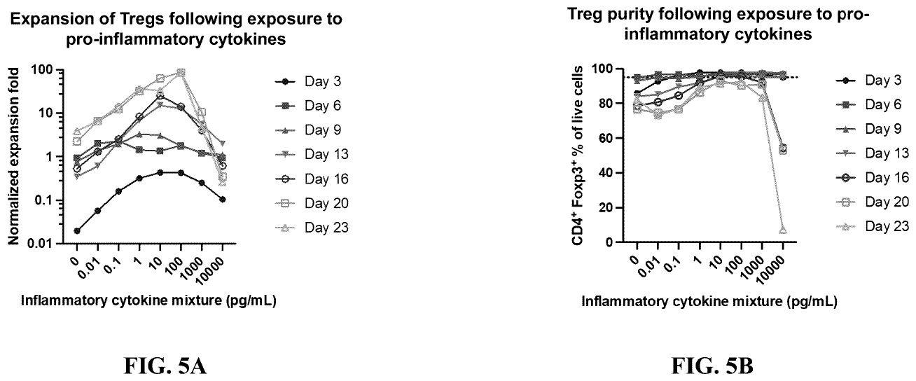

The expansion and Treg purity were measured over a time-course ranging from 3-23 days in the presence of IL-2 and all 8 cytokines, each at the concentration shown. The data show that the regulatory T cells continued to expand and remained stable for up to 23 days when cultured in the presence of the cytokine mixture of 1000 U/mL IL-2 and up to 1000 pg/mL of each of IL-1β, IL-6, IL-12, IL-17A, IFN-β, IFN-γ, TGF-β1, and TNF-α (FIGS. 5A-5B).

### Example 2

The ability of a population of engineered regulatory T cells to expand in the presence of the combination of IL-2 and TNF-α was further evaluated with expansion out to day 12. Previously expanded and frozen CD4+CD25+CD127lowFoxP3+T cell receptor (TCR)-transduced regulatory T cells were thawed and plated in T cell culture media (RPMI 1640 based medium supplemented with 10%, serum, 100 U/mL Pen/Strep, non-essential amino acids, and sodium pyruvate). Cells were cultured for twelve days in the presence of CD3/CD28 Dynabeads and either (1) IL-2 alone (1000 U/mL), or (2) IL-2 at 1000 U/mL and 1000 pg/mL of TNF-α.

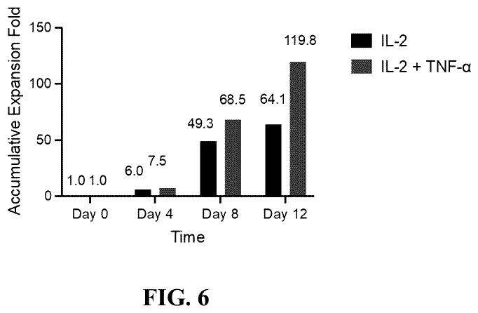

The fold expansion of the cell populations (relative to Day 0) was measured on Days 4, 8, and 12. It was shown that the population of regulatory T cells grown in the presence of IL-2 and TNF-α was expanded by almost 120-fold over the period of twelve days (FIG. 6). Conversely, the population of regulatory T cells grown in the presence of IL-2 alone was expanded by only 64-fold over the period of twelve days.

These data demonstrate that culturing a population of regulatory T cells grown in the presence of IL-2 and TNF-α provides a significant advantage in expansion and proliferation relative to the use of IL-2 alone.

### Example 3

In this Example, the phenotypic stability of engineered regulatory T cells after exposure to inflammatory stimuli was evaluated. Highly pure CD4+CD25+CD127^{low}WFoxP3⁺ T cell receptor (TCR)-transduced regulatory T cells having at least 88% of cells hypomethylated at the TSDR locus were thawed and plated at 50,000 cells per well in a 96-well plate in triplicate in X-VIVO15 medium. Cells were cultured for three days in the presence of CD3/CD28 Dynabeads™ and IL-2 (1000 U/mL), or CD3/CD28 Dynabeads™ and one of two different mixtures of pro-inflammatory cytokines: (1) IL-2 at 1000 U/mL and 1000 pg/mL of each of IL-1β, IL-6, IL-12, IL-17A, IFN-γ, and TNF-α (Mix-6); or (2) IL-2 at 1000 U/mL and 1000 pg/mL of each of IL-1β, IL-6, IL-12, IL-17A, IFN-γ, TNF-α, IFN-β, and TGF-β (Mix-8). The engineered regulatory T cells were exposed to control, Mix-6, or Mix-8 treatment over a period of 3 days.

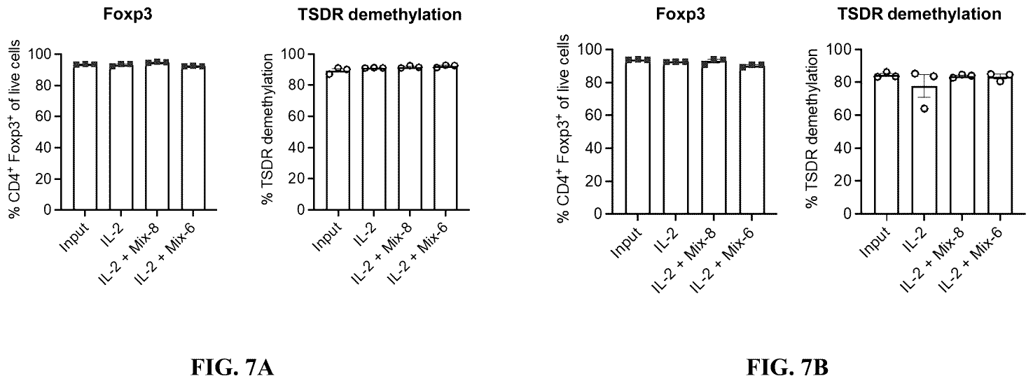

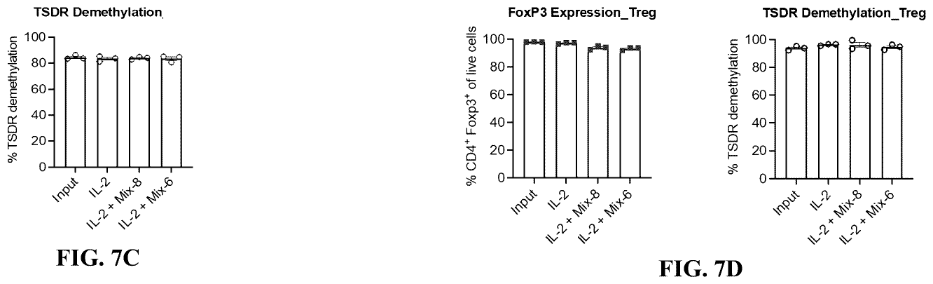

FACS analysis to determine FOXP3 expression and cell number and TSDR analysis before and after culture in the mixture of pro-inflammatory cytokines from three different donors showed that the regulatory T cells are stable after isolation, expansion, freeze-thaw and exposure to inflammatory cytokines (FIGS. 7A, 7B and 7D). FIG. 7C is a repeat of the TSDR assay of FIG. 7B done using an alternative bisulfite conversion method specifically designed for low cell counts.

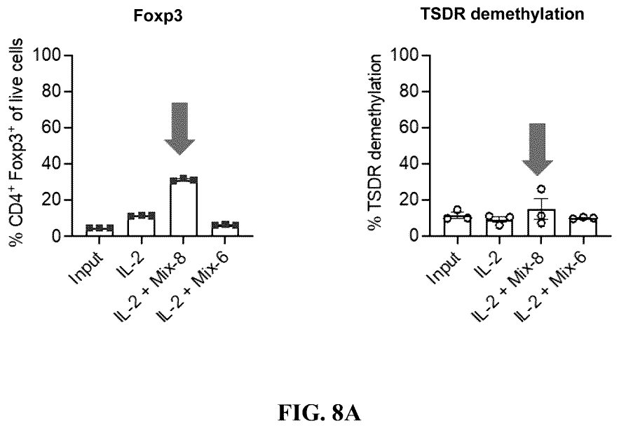

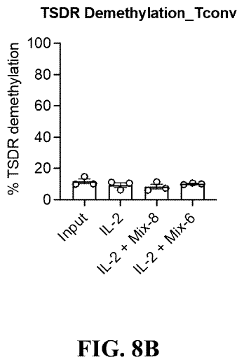

Conventional T cells from the donor shown in FIG. 7D were similarly cultured in the presence of IL-2 or one of the two different mixtures of pro-inflammatory cytokines (with CD3/CD28 Dynabeads™ in each instance). FACS and TSDR analysis showed that, in Mix-8, the conventional T cells are induced to express low levels of FOXP3, but TSDR stays hypomethylated, distinct from the stable regulatory T cells shown in FIG. 7 (FIG. 8A). FIG. 8B is a repeat of the TSDR assay of FIG. 7A done using an alternative bisulfite conversion method specifically designed for low cell counts.

### Example 4

FOXP3 expression can be induced in conventional CD4+ T cells in cell culture using TGF-β. These T cells expressing high levels of FOXP3 induced by TGF-β are referred to as induced Tregs or iTregs and are capable of suppressing effector cell function. However, these iTregs are inherently unstable and removal of TGF-β can lead to the downregulation of FOXP3 and the reestablishment of their proinflammatory effector functions. The rationale of this study is to show that unstable “iTregs” (in vitro induced Tregs grown primarily from Tconv cells under cytokine skewing conditions) downregulate FOXP3 and reestablish effector functions in a matter of days in the absence of TGF-β, while “Tregs” (thymus educated Tregs that have undergone development in the thymus and stably express FOXP3 and have a hypomethylated TSDR) maintain FOXP3 expression during this same period of time and do not function like effector T cells. The experiments described in this example demonstrate that unstable iTregs are evident in a 5-day assay and that a representative stable Treg product shows no evidence of instability in that time frame.

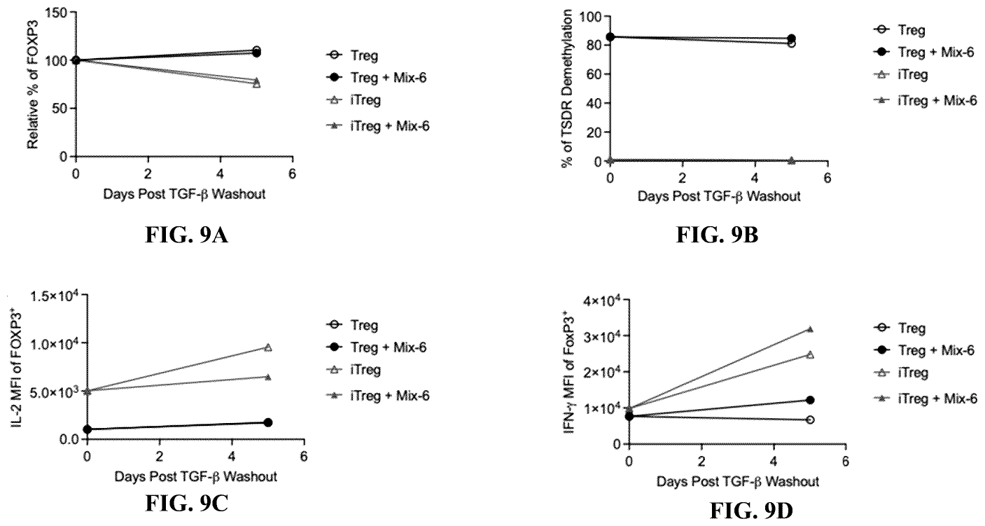

In the following experiment, test material was made by sorting CD4⁺ T cells and dividing the cells into CD25^{hi}CD127^{low} Tregs (Tregs) and CD25^{low} Tconv cells. T conventional cells were cultured with both IL-2 and TGF-β to induce iTregs for 7 days. In parallel, Tregs were cultured in the presence of IL-2 for only. After 7 days in culture for expansion of Tregs and induction of iTregs, the cells were collected and washed to remove residual cytokine TGF-β and IL-2. The cells were then placed in culture with either IL-2 alone or in the presence of IL-2 with Mix-6. On day 3, cells were fed with fresh media supplemented with IL-2. FOXP3 protein expression (FIG. 9A) and TSDR demethylation (FIG. 9B).

A proportion of the cells were stimulated with phorbol 12-myristate 13-acetate (PMA) and ionomycin for 4 hours 5 days after the addition of inflammatory cytokines. This proportion of cells was then stained for the presence of intracellular IL-2 (FIG. 9C) and IFN-γ (FIG. 9D) as readouts of effector cell function.

The data show that at the end of the initial 7-day Treg expansion or iTreg induction, both Tregs and iTregs expressed high levels of FOXP3. After removal of TGF-β, Tregs maintained FOXP3 expression while the iTregs downregulated FOXP3 (Error! Reference source not found. 9A). Demethylation of the TSDR was only detected in Tregs and remained stable over the course of 5 days (FIG. 9B). When stimulated with PMA and ionomycin the iTreg cultures produced higher levels of IL-2 and IFN-γ (FIG. 9C and FIG. 9D). Tregs cultured in the presence of pro-inflammatory cytokines, maintained low effector function as measured by IL-2 and IFN-γ production. This data shows iTregs quickly lose their Treg phenotype, and this is observable within 5-days, while Tregs are stable, even in the presence of inflammatory cytokines. This supports the hypothesis that Treg instability is observed within the iTreg population, and this instability can emerge within 5 days of culture.

### Example 5

In this Example, the phenotypic stability of engineered regulatory T cells in vitro after exposure to inflammatory stimuli for five days was evaluated (FIG. 1).

CD4₊ CD25⁺ CD127^{low} regulatory T cells (Tregs) were isolated from PBMCs belonging to a healthy human donor. Isolated Tregs were expanded for 12 days with lentiviral transduction to express a representative TCR. During the transduction and expansion phase, Tregs were cultured with anti-CD3/CD28 antibody coated microbeads and recombinant human IL-2. After a cryopreservation and cell thawing cycle, the engineered Tregs (expressing the exogenous TCR) were then subjected to the method described in Example 1 to determine the stability profile of the engineered Tregs. Specifically, the engineered Tregs were cultured in the presence of (1) IL-2 alone, (2) IL-2 and a mixture of proinflammatory cytokines including IL-1β, IL-6, IL-12 p70, IL-17A, IFN-β, IFN-γ, TGF-β, and TNF-α (Mix-8), or (3) IL-2 and a mixture of IL-1β, IL-6, IL-12 p70, IL-17A, IFN-β, and IFN-γ (Mix-6) for 5 days. Non-lentiviral transduced but expanded CD4⁺ CD25^{low} CD127^{high} conventional T cells and CD4⁺ CD25⁺ CD127^{low} Tregs were used as control experiments using the same set of culture conditions.

In this assay, the cell populations were cultured for a total of 5 days after a thaw. Cytokines were added on the first day of culturing post-thaw (i.e., Day 0) and on Day 3. TSDR demethylation, FOXP3 protein expression, and the production of intracellular proinflammatory cytokine IFN-γ were measured on Day 0 and again on Day 5 (after activation with PMA and ionomycin for 4 hours).

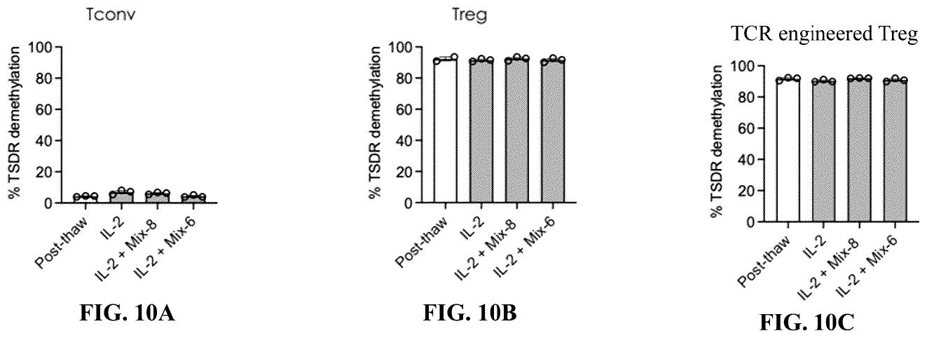

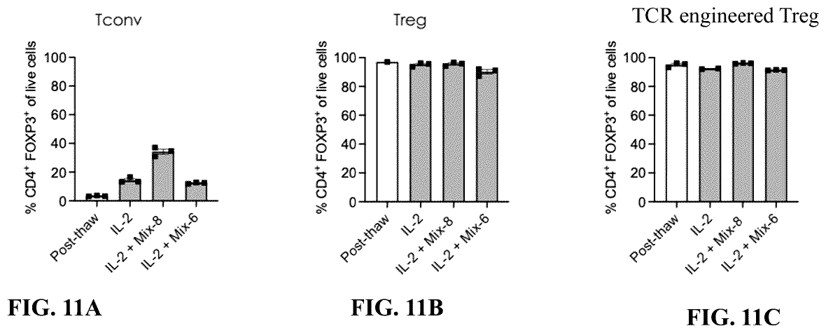

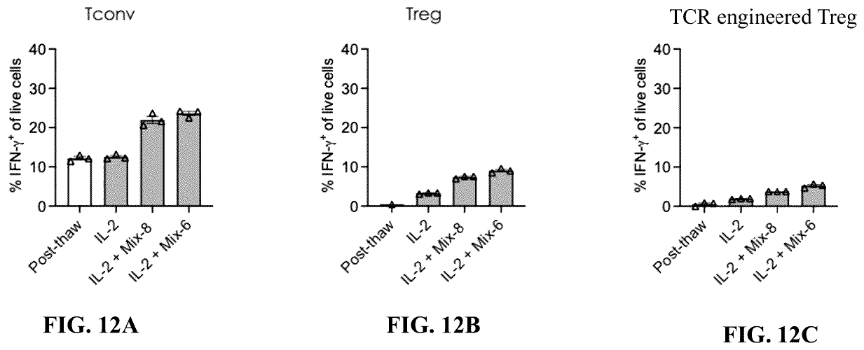

The data demonstrates that engineered Tregs (“TCR engineered Treg” populations) are phenotypically stable over 5 days when treated with proinflammatory cytokines, as TSDR demethylation status (>90% in total cell pellets (FIG. 10C) and FOXP3 expression (>90% of CD4+ FOXP3+ cells among total live cells (FIG. 11C)) were maintained at Day 5, with minimal levels of IFN-γ expression (<10% among total live cells (FIG. 12C)). The untransduced Tregs (“Treg” populations) demonstrated similar phenotypic stability profiles as compared to the engineered Tregs (FIGS. 10B, 11B, and 12B). Conversely, the data demonstrates that Tconv cells (“Tconv” populations) were not phenotypically stable with induced FOXP3 expression (up to 34.2%, FIG. 11A) and significantly upregulated IFN-γ production (up to 32.0%, FIG. 12A), which suggests that the proinflammatory conditions used in the study exerted significant impact on the phenotype of the Tconv, and that this was observable in the 5-day duration of the assay. Additionally, Tconv cells had consistently low level TSDR demethylation (<7.0%, FIG. 10A) even in the conditions that provided induced expression of FOXP3 and IFN-γ, indicating that the TSDR demethylation shown in Tregs and engineered Tregs did not result from induction of FOXP3.

These data demonstrate that regulatory T cells (e.g., engineered regulatory T cells) are stable under inflammatory conditions (such as inflammatory conditions that approximate the pro-inflammatory environment within a subject who has been administered the regulatory T cells) for a period of time (e.g., at least five days). Furthermore, these data demonstrate that the stability profile of a cell population comprising regulatory T cells (e.g., engineered regulatory T cells) can be successfully determined using an in vitro method of culturing said cells in the presence of two or more pro-inflammatory cytokines (e.g., two or more cytokines selected from IL-2, IL-1β, IL-6, IL-12 p70, IL-17A, IFN-β, IFN-γ, TGF-β, and TNF-α) and characterizing markers of stability of the cells (e.g., FOXP3 expression, TSDR demethylation, and IFN-γ production).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of evaluating the stability profile of an isolated cell population comprising regulatory T cells, the method comprising:
(i) culturing the isolated cell population in the presence of two or more pro-inflammatory cytokines for a period of time;
(ii) characterizing one or more markers of stability of the isolated cell population following the period of time; and
(iii) evaluating the stability profile of the isolated cell population based on the characterizing of the one or more markers of stability.

2. A method comprising:
(i) culturing an isolated cell population comprising regulatory T cells in the presence of two or more pro-inflammatory cytokines for a period of time; and
(ii) characterizing one or more markers of stability of the isolated cell population following the period of time.

3. The method of claim 1, wherein the two or more pro-inflammatory cytokines comprises at least two, at least three, at least four, at least five, at least six, at least seven, at least eight, or at least nine cytokines selected from the group consisting of Interleukin-2 (IL-2), Interleukin-6 (IL-6), Interleukin-12 (IL-12), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

4. The method of claim 1, wherein the two or more pro-inflammatory cytokines comprises IL-2 and at least one, at least two, at least three, at least four, at least five, at least six, at least seven, or at least eight cytokines selected from the group consisting of IL-6, IL-12, TNF-α, IL-17A, IFN-β, IFN-γ, TGF-β1, and IL-1β.

5. The method of claim 1, wherein the two or more pro-inflammatory cytokines comprises:
(a) IL-2 and IL-6;
(b) IL-2 and IL-12;
(c) IL-2 and TNF-α;
(d) IL-2 and IL-17A;
(e) IL-2 and IFN-β;
(f) IL-2 and IFN-γ;
(g) IL-2 and TGF-β1; or
(h) IL-2 and IL-1β.

6. The method of claim 1, wherein the two or more pro-inflammatory cytokines comprises:
(a) IL-2, IL-6, and at least one additional pro-inflammatory cytokine selected from: IL-12, TNF-α, IL-17A, IFN-β, IFN-γ, TGF-β1, and IL-1β;
(b) IL-2, IL-12, and at least one additional pro-inflammatory cytokine selected from: IL-6, TNF-α, IL-17A, IFN-β, IFN-γ, TGF-β1, and IL-1β;
(c) IL-2, TNF-α, and at least one additional pro-inflammatory cytokine selected from: IL-6, IL-12, IL-17A, IFN-β, IFN-γ, TGF-β1, and IL-1β;
(d) IL-2, IL-17A, and at least one additional pro-inflammatory cytokine selected from: IL-6, IL-12, TNF-α, IFN-β, IFN-γ, TGF-1, and IL-1β;
(e) IL-2, IFN-β, and at least one additional pro-inflammatory cytokine selected from: IL-6, IL-12, TNF-α, IL-17A, IFN-γ, TGF-β1, and IL-1β;
(f) IL-2, IFN-γ, and at least one additional pro-inflammatory cytokine selected from: IL-6, IL-12, TNF-α, IL-17A, IFN-β, TGF-β1, and IL-1β;
(g) IL-2, TGF-β1, and at least one additional pro-inflammatory cytokine selected from: IL-6, IL-12, TNF-α, IL-17A, IFN-β, IFN-γ, and IL-1β; or
(h) IL-2, IL-1β, and at least one additional pro-inflammatory cytokine selected from: IL-6, IL-12, TNF-α, IL-17A, IFN-β, IFN-γ, and TGF-β1.

7. The method of claim 1, wherein the two or more pro-inflammatory cytokines comprises:
(a) IL-2, IL-6, and at least two additional pro-inflammatory cytokines selected from: IL-12, TNF-α, IL-17A, IFN-β, IFN-γ, TGF-β1, and IL-1β;
(b) IL-2, IL-12, and at least two additional pro-inflammatory cytokines selected from: IL-6, TNF-α, IL-17A, IFN-β, IFN-γ, TGF-β1, and IL-1β;
(c) IL-2, TNF-α, and at least two additional pro-inflammatory cytokines selected from: IL-6, IL-12, IL-17A, IFN-β, IFN-γ, TGF-β1, and IL-1β;
(d) IL-2, IL-17A, and at least two additional pro-inflammatory cytokines selected from: IL-6, IL-12, TNF-α, IFN-β, IFN-γ, TGF-β1, and IL-1β;
(e) IL-2, IFN-β, and at least two additional pro-inflammatory cytokines selected from: IL-6, IL-12, TNF-α, IL-17A, IFN-γ, TGF-β1, and IL-1β;
(f) IL-2, IFN-γ, and at least two additional pro-inflammatory cytokines selected from: IL-6, IL-12, TNF-α, IL-17A, IFN-β, TGF-β1, and IL-1β;
(g) IL-2, TGF-β1, and at least two additional pro-inflammatory cytokines selected from: IL-6, IL-12, TNF-α, IL-17A, IFN-β, IFN-γ, and IL-1β; or
(h) IL-2, IL-1β, and at least two additional pro-inflammatory cytokines selected from: IL-6, IL-12, TNF-α, IL-17A, IFN-β, IFN-γ, and TGF-β1.

8. The method of claim 1, wherein the two or more pro-inflammatory cytokines comprises:
(a) IL-2, IL-6, and at least three additional pro-inflammatory cytokines selected from: IL-12, TNF-α, IL-17A, IFN-β, IFN-γ, TGF-β1, and IL-1β;
(b) IL-2, IL-12, and at least three additional pro-inflammatory cytokines selected from: IL-6, TNF-α, IL-17A, IFN-β, IFN-γ, TGF-β1, and IL-1β;
(c) IL-2, TNF-α, and at least three additional pro-inflammatory cytokines selected from: IL-6, IL-12, IL-17A, IFN-β, IFN-γ, TGF-β1, and IL-1β;
(d) IL-2, IL-17A, and at least three additional pro-inflammatory cytokines selected from: IL-6, IL-12, TNF-α, IFN-β, IFN-γ, TGF-β1, and IL-1β;
(e) IL-2, IFN-β, and at least three additional pro-inflammatory cytokines selected from: IL-6, IL-12, TNF-α, IL-17A, IFN-γ, TGF-β1, and IL-1β;
(f) IL-2, IFN-γ, and at least three additional pro-inflammatory cytokines selected from: IL-6, IL-12, TNF-α, IL-17A, IFN-β, TGF-β1, and IL-1β;
(g) IL-2, TGF-β1, and at least three additional pro-inflammatory cytokines selected from: IL-6, IL-12, TNF-α, IL-17A, IFN-β, IFN-γ, and IL-1β; or
(h) IL-2, IL-1β, and at least three additional pro-inflammatory cytokines selected from: IL-6, IL-12, TNF-α, IL-17A, IFN-β, IFN-γ, and TGF-β1.

9. The method of claim 1, wherein the two or more pro-inflammatory cytokines comprises IL-2, IL-6, IL-12, TNF-α, IL-17A, IFN-β, IFN-γ, TGF-β1, and IL-1β.

10. The method of claim 1, wherein the two or more pro-inflammatory cytokines comprise IL-2, IL-1β, IL-6, IL-12, IL-17A, IFN-γ, TNF-α, IFN-β, and TGF-β1, optionally wherein the concentration of each of IL-1β, IL-6, IL-12, IL-17A, IFN-γ, TNF-α, IFN-β, and TGF-β1 is about 1000 pg/mL, optionally wherein the concentration of IL-2 is 1000 U/mL.

11. The method of claim 1, wherein the two or more pro-inflammatory cytokines comprise IL-2, IL-1β, IL-6, IL-12, IL-17A, IFN-γ, and TNF-α, optionally wherein the concentration of each of IL-1β, IL-6, IL-12, IL-17A, IFN-γ, and TNF-α is about 1000 pg/mL, optionally wherein the concentration of IL-2 is 1000 U/mL.

12. The method of claim 1, wherein the two or more pro-inflammatory cytokines further comprise Interleukin-22 (IL-22), Granulocyte-macrophage colony-stimulating factor (GM-CSF) and/or Interleukin-23 (IL-23).

13. The method of claim 1, wherein the concentration of any one of the pro-inflammatory cytokines is in the range of 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

14. The method of 3, wherein the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL.

15. The method of claim 1, wherein the period of time is at least 3, at least 6, at least 9, at least 12, at least 15, at least 20, or at least 25 days.

16. The method of claim 1, wherein the period of time is 3 days, 4 days, 5 days, 6 days, 7 days, 8 days, or 9 days.

17. The method of claim 1, wherein the period of time is 2-30 days, 3-25 days, 5-25 days, 5-20 days, 5-10 days, 10-30 days, 10-25 days, 10-20 days, 15-30 days, or 20-30 days.

18. The method of claim 1 further comprising characterizing the one or more markers of stability of the isolated cell population prior to (i).

19. The method of claim 1 further comprising activating the isolated cell population between steps (i) and (ii), optionally wherein activating the isolated cell population is performed by incubating the cell population with phorbol 12-myristate 13-acetate (PMA) and ionomycin.

20. The method of claim 1 further comprising characterizing the one or more markers of stability of the isolated cell population at regular intervals throughout the period of time.

21. The method of claim 20, wherein the method comprises characterizing the one or more markers of stability of the isolated cell population every day, every second day, every third day, or every fourth day throughout the period of time.

22. The method of claim 1, wherein characterizing one or more markers of stability comprises determining expression and/or activity of the one or more markers of stability.

23. The method of claim 22, wherein determining expression and/or activity of the one or more markers of stability is performed using biochemical techniques.

24. The method of claim 22, wherein determining expression and/or activity of the one or more markers of stability is performed using fluorescence activated cell sorting (FACS).

25. The method of claim 1, wherein the one or more markers of stability comprise the biomarkers CD4, CD25, and/or FOXP3.

26. The method of claim 1, wherein the one or more markers of stability comprise the methylation of a regulatory T cell-specific demethylation region (TSDR) at an endogenous FOXP3 locus and/or FOXP3 expression.

27. The method of claim 1 further comprising characterizing the relative abundance of IFN-γ⁺ cells, IL-¹⁷⁺ cells, and/or TNF-α⁺ cells within the cell population after (i).

28. The method of claim 1, wherein at least 80% of the cells of the isolated cell population are CD25^{+/high}CD4⁺CD127^{-/lo} regulatory T cells.

29. The method of claim 1, wherein, prior to the culturing of step (i), at least 70% of the cells of the isolated cell population are stable regulatory T cells comprising a hypomethylated TSDR at an endogenous FOXP3 locus and/or at least 80% of the cells of the isolated cell population are FOXP3+.

30. The method of claim 1, wherein the isolated cell population has undergone one or more cryopreservation freeze-thaw cycles.

31. The method of claim 1, wherein culturing the isolated cell population comprises culturing the isolated cell population in a cell media.

32. The method of claim 31, wherein culturing the isolated cell population comprises replacing the cell media with new cell media at regular intervals.

33. The method of claim 31, wherein the cell media comprises a buffer.

34. The method of claim 31, wherein the cell media comprises phosphate-buffered saline (PBS), bovine serum albumin, human serum albumin, and/or EDTA.

35. The method of claim 1, wherein the isolated cell population is cultured in the presence of one or more agents that activate T cells within the isolated cell population, optionally wherein the one or more agents are antigen-presenting cells or agents that mimic antigen-presenting cells.

36. The method of claim 35, wherein the agents that mimic antigen-presenting cells are three-dimensional beads, optionally wherein the three-dimensional beads are attached to activation signals.

37. The method of claim 35, wherein the agents that mimic antigen-presenting cells are three-dimensional beads that are attached to anti-CD3, anti-CD28, and/or anti-CD137 molecules, optionally wherein the agents are polystyrene beads coated with a mixture of anti-CD3 and/or anti-CD28 antibodies.

38. The method of claim 35, wherein the one or more agents that activate T cells are phorbol 12-myristate 13-acetate (PMA) and ionomycin.

39. The method of claim 31, wherein culturing the isolated cell population comprises replacing the cell media with new cell media every 2-5 days, optionally every 3-4 days.

40. The method of claim 1, wherein the isolated cell population is a stable cell population if at least 80% of the regulatory T cells comprise a hypomethylated TSDR and an endogenous FOXP3 locus and/or at least 80% of the cells of the isolated cell population are FOXP3+.

41. A method of expanding a cell population, the method comprising culturing an isolated cell population comprising regulatory T cells in the presence of IL-2 and at least three additional pro-inflammatory cytokines.

42. The method of claim 41, wherein the at least three additional pro-inflammatory cytokines are selected from the group consisting of Interleukin-6 (IL-6), Interleukin-12 (IL-12), Tumor necrosis factor (TNF-α), Interleukin-17 (IL-17A), Interleukin-21 (IFN-β), Interferon gamma (IFN-γ), Transforming growth factor beta 1 (TGF-β1), and Interleukin-1b (IL-1β).

43. The method of claim 41, wherein the at least three additional pro-inflammatory cytokine comprises at least four, at least five, at least six, at least seven, or at least eight additional pro-inflammatory cytokines selected from the group consisting of Interleukin-6 (IL-6), Interleukin-12 (IL-12), Tumor necrosis factor (TNF-α), Interleukin-17 (IL-17A), Interferon beta (IFN-β), Interferon gamma (IFN-γ), Transforming growth factor beta 1 (TGF-β1), and Interleukin-1b (IL-1β).

44. The method of claim 41, wherein the at least three additional pro-inflammatory cytokines comprise IL-6, IL-12, TNF-α, IL-17A, IFN-β, IFN-γ, TGF-β1, and IL-1β.

45. A method of expanding a cell population, the method comprising culturing an isolated cell population comprising regulatory T cells in the presence of Interleukin-17 (IL-17A).

46. A method of expanding a cell population, the method comprising culturing an isolated cell population comprising regulatory T cells in the presence of Interferon beta (IFN-β).

47. A method of expanding a cell population, the method comprising culturing an isolated cell population comprising regulatory T cells in the presence of Interferon gamma (IFN-γ).

48. A method of expanding a cell population, the method comprising culturing an isolated cell population comprising regulatory T cells in the presence of Interleukin-1b (IL-1β).

49. A method of expanding a cell population, the method comprising culturing an isolated cell population comprising regulatory T cells in the presence of Tumor necrosis factor alpha (TNF-α) and IL-2.

50. The method of claim 45 further comprising culturing the isolated cell population in the presence of one or more additional pro-inflammatory cytokines.

51. The method of claim 50, wherein the one or more additional pro-inflammatory cytokines is Interleukin-2 (IL-2), Interleukin-6 (IL-6), Interleukin-12 (IL-12), Tumor necrosis factor alpha (TNF-α), Interleukin-17 (IL-17A), Interferon gamma (IFN-γ), Interferon beta (IFN-β), Transforming growth factor beta 1 (TGF-β1), and Interleukin-1 beta (IL-1β).

52. The method of claim 41, wherein the concentration of any one of the pro-inflammatory cytokines is in the range of 0.01-10000 pg/mL, 0.01-10000 pg/mL, 0.01-1000 pg/mL, 0.01-100 pg/mL, 0.01-10 pg/mL, 0.01-1 pg/mL, 1-1000 pg/mL, 1-500 pg/mL, 1-100 pg/mL, 10-1000 pg/mL, 10-100 pg/mL, 50-500 pg/mL, 100-500 pg/mL, 100-750 pg/mL, 100-1000 pg/mL, 250-1000 pg/mL, 500-1000 pg/mL, 500-10000 pg/mL, 500-5000 pg/mL, 1000-10000 pg/mL, 1000-5000 pg/mL, or 100-10000 pg/mL.

53. The method of claim 41, wherein the concentration of IL-2 is in the range of 1-10000 U/mL, 1-7500 U/mL, 1-5000 U/mL, 1-2500 U/mL, 1-1000 U/mL, 100-5000 U/mL, 100-2500 U/mL, 100-1000 U/mL, 100-500 U/mL, 250-1000 U/mL, 250-2500 U/mL, 500-5000 U/mL, 500-2500 U/mL, 500-1000 U/mL, or 750-2000 U/mL, optionally wherein the concentration of IL-2 is 1000 U/mL.

54. The method of claim 41, wherein the concentration of each of the at least three additional pro-inflammatory cytokines is in the range of 100-1000 pg/mL.

55. The method of claim 41, wherein the pro-inflammatory cytokines induce a signaling cascade within the regulatory T cells of the isolated cell population.

56. The method of claim 41, wherein the level of expansion of the isolated cell population is determined relative to a control, optionally wherein the control is a control isolated cell population that is not being cultured in the presence of additional pro-inflammatory cytokines.

57. The method of claim 56, wherein the level of expansion of the isolated cell population is increased relative to the control.

58. The method of claim 41, wherein at least 80% of the cells of the isolated cell population are CD25^{+/high}CD4⁺CD127^{-/lo} regulatory T cells.

59. The method of claim 41, wherein the isolated cell population is a stable cell population if at least 80% of the regulatory T cells comprise a hypomethylated TSDR and an endogenous FOXP3 locus.

60. The method of claim 41, wherein the isolated cell population has undergone one or more cryopreservation freeze-thaw cycles prior to culturing.

61. The method of claim 41, wherein culturing the isolated cell population comprises culturing the isolated cell population in a cell media.

62. The method of claim 61, wherein culturing the isolated cell population comprises replacing the cell media with new cell media at regular intervals.

63. The method of claim 61, wherein culturing the isolated cell population comprises replacing the cell media with new cell media every 2-5 days, optionally every 3-4 days.

64. The method of claim 41, wherein culturing the isolated cell population comprises culturing the isolated cell population for at least 3, at least 6, at least 9, at least 12, at least 15, at least 20, or at least 25 days.

65. The method of claim 41, wherein culturing the isolated cell population comprises culturing the isolated cell population for 2-30 days, 3-25 days, 5-25 days, 5-20 days, 5-10 days, 10-30 days, 10-25 days, 10-20 days, 15-30 days, or 20-30 days.

66. The method of claim 41, further comprising isolating the cell population.

67. The method of claim 61, wherein the cell media comprises a buffer.

68. The method of claim 61, wherein the cell media comprises phosphate-buffered saline (PBS), bovine serum albumin, human serum albumin, and/or EDTA.

69. The method of claim 41, wherein the isolated cell population is cultured in the presence of one or more agents that activate T cells within the isolated cell population, optionally wherein the one or more agents are antigen-presenting cells or agents that mimic antigen-presenting cells.

70. The method of claim 69, wherein the agents that mimic antigen-presenting cells are three-dimensional beads, optionally wherein the three-dimensional beads are attached to activation signals.

71. The method of claim 69, wherein the agents that mimic antigen-presenting cells are three-dimensional beads are attached to anti-CD3, anti-CD28, and/or anti-CD137, optionally wherein the agents are polystyrene beads coated with a mixture of anti-CD3 and/or anti-CD28 antibodies.
